(19) United States
(12) Patent Application Publication
(10) Pub. No.: US 2021/0401768 A1
(43) Pub. Date: Dec. 30, 2021

(54) FORMULATIONS OF CANNABINOIDS FOR THE TREATMENT OF DERMATITIS AND INFLAMMATORY SKIN DISEASES

(71) Applicants: Botanix Pharmaceuticals Ltd., Northbridge (AU); Botanix Pharmaceuticals, Inc., Plymouth Meeting, PA (US)

(72) Inventors: Eugene R. Cooper, Berwyn, PA (US); Matthew Callahan, Philadelphia, PA (US)

(21) Appl. No.: 16/486,290

(22) PCT Filed: Jan. 24, 2018
(86) PCT No.: PCT/AU2018/050044

§ 371 Date: Aug. 15, 2019

Related U.S. Application Data
(60) Provisional application No. 62/459,363, filed on Feb. 15, 2017.

(30) Foreign Application Priority Data
Feb. 15, 2017 (AU) 2017900495

(51) Int. Cl. A61K 31/05; A61K 9/08; A61K 47/24; A61K 9/00; A61P 17/00; A61P 29/00

(52) U.S. Cl. CPC A61K 31/05; A61K 9/08; A61P 29/00; A61K 9/0014; A61P 17/00; A61K 47/24

(57) ABSTRACT

A pharmaceutical composition comprising a cannabinoid and a siloxane wherein the cannabinoid is dissolved in the composition.

### TECHNICAL FIELD

The present invention relates to a pharmaceutical composition for the delivery of a cannabinoid. The pharmaceutical composition of the present invention is particularly suited for the treatment of inflammatory skin conditions.

### BACKGROUND ART

The following discussion of the background art is intended to facilitate an understanding of the present invention only. The discussion is not an acknowledgement or admission that any of the material referred to is or was part of the common general knowledge as at the priority date of the application.

Most mammalian skin, including human skin, comprises three layers: (i) an epidermis layer, which is predominantly composed of keratinocytes and a small number of melanocytes and Langerhans cells (antigen presenting cells); (ii) a dermis layer, which contains nerve endings, sweat glands and oil (sebaceous) glands, hair follicles, and blood vessels and which is primarily composed of fibroblasts; and (iii) a hypodermis layer of deeper subcutaneous fat and connective tissue. The epidermis itself is made up of two layers, the outer stratum corneum and the inner epidermal basal layer.

The majority of skin conditions involve inflammation triggered by some insult to the skin. Keratinocytes respond quickly to environmental stimuli (e.g., UV radiation (UVR), allergens, irritants or physical damage) by producing a variety of inflammatory mediators, including cytokines (e.g., IL-I, TNF-alpha, and IL-6) and chemokines (e.g., IL-8). One of the most active inflammatory mediators is PGE-2 (Prostaglandin E2) and, of course, many topical dermatology drugs have been designed to lower levels of PGE-2. The fibroblasts in the dermis also produce PGE-2 along with a variety of chemokines, cytokines and matrix destroying enzymes such as collagenase (MMP-I).

Eczema, also known as dermatitis, is a general term for many types of skin conditions that involve inflammation. Atopic dermatitis is the most common of the many types of eczema. Several other forms have very similar symptoms. Some of the diverse types of eczema are listed and briefly described below.

Atopic dermatitis is a chronic skin disease wherein the skin becomes extremely itchy and inflamed, causing redness, swelling, cracking, weeping, crusting, and scaling. Atopic dermatitis most often affects infants and young children, but it can continue into adulthood or first show up later in life. Onset after age 30 is less common and often occurs after exposure of the skin to harsh conditions. In most cases, there are periods of time when the disease is worse, called exacerbations or flares, which are followed by periods when the skin improves or clears up entirely, called remissions. The cause of atopic dermatitis is unknown, but the disease seems to result from a combination of genetic and environmental factors. Atopic dermatitis is very common and affects males and females equally and accounts for 10 to 20% of all referrals to dermatologists; more than 15 million people in the United States have symptoms of the disease. People who live in urban areas and in climates with low humidity seem to be at an increased risk for developing atopic dermatitis.

Contact eczema is a localized reaction that includes redness, itching, and burning where the skin has come into contact with an allergen (an allergy-causing substance) or with an irritant such as an acid, a detergent (soap, bodywash), or other chemical.

Allergic contact eczema is a red, itchy, weepy reaction where the skin has come into contact with a substance that the immune system recognizes as foreign, such as poison ivy or certain preservatives in creams and lotions.

Seborrheic eczema is a form of skin inflammation of unknown cause but which is associated with a certain type of yeast that lives on the skin. Seborrheic eczema presents as yellowish, oily, scaly patches of skin on the scalp, face, and occasionally other parts of the body (called cradle cap in infants).

Nummular eczema is coin-shaped patches of irritated skin—most commonly on the arms, back, buttocks, and lower legs—that may be crusted, scaling, and extremely itchy.

Neurodermatitis is scaly patches of skin on the head, lower legs, wrists, or forearms caused by a localized itch (such as an insect bite) that becomes intensely irritated when scratched.

Stasis dermatitis is a skin irritation on the lower legs, generally related to circulatory problems.

Dyshidrotic eczema is irritation of the skin on the palms of hands and soles of the feet characterized by clear, deep blisters that itch and burn.

Radiation therapy can have some unpleasant side effects which include inflammation of the skin and radiation dermatitis. Specific side effects of radiotherapy, both acute and chronic, depend on the part of the body being treated as well as the dose given. In general, the first change is a reddening of the skin, resembling sunburn. In many patients this is all that is experienced. However, in most patients the burn can be severe and in many cases equivalent to second degree burns. Like sunburn, the involved area is often sensitive and even painful to the touch. In addition, the overlying skin may break down and the area may remain open until several days to weeks after the course of radiation is completed. Once the course of radiotherapy is completed, the redness will gradually go away and any open areas normally will heal. However, the skin in this area will most likely develop features of aged skin including pronounced wrinkling, skin thinning, stiffness and/or dryness, as well as possible pigmentation changes.

Most of the current treatment options for radiation dermatitis involve the use of emollients or aloe gels in an attempt to keep the skin moisturized. However, although moisturization helps the skin from drying out, it does not reduce the pain or redness, which are caused by inflammation.

Rosacea is a vascular, inflammatory skin disorder that affects approximately 5% of the population and is characterized by frequent periods of facial redness or flushing caused by over-active capillaries. Over time, this chronic state of skin inflammation gives rise to a variety of rosacea symptoms. Rosacea is sometimes characterized mistakenly as adult-acne because patients present with a reddened face and acne-like symptoms. However, individuals affected with this skin disease also may have persistent redness with accompanying pain and itching in areas such as the forehead, chin, nose, ears, chest and back. As the disease progresses, small blood vessels and tiny pimples (called papules or pustules) begin to appear on and around the reddened area. In severe cases rosacea can affect the eyes (ocular rosacea) and cause disfigurement of the nose (rhynophyma). In addition to the physical symptoms associated with rosacea, patients also suffer significant psychological and social problems if left untreated.

The present invention seeks to provide a composition and method to reduce the effects of the conditions mentioned above and other inflammatory skin conditions, or to provide the consumer with a useful or commercial choice.

### SUMMARY OF INVENTION

In accordance with the present invention, there is provided a pharmaceutical composition comprising a cannabinoid and a siloxane wherein the cannabinoid is dissolved in the composition. In accordance with one embodiment, the cannabinoid is cannabidiol. In accordance with another aspect of the invention, the pharmaceutical composition is a topical pharmaceutical composition. The siloxane forms a volatile solvent for the cannabinoid.

The cannabinoids delivered by the present invention preferably penetrate into the epidermis of the skin, and most of the cannabinoids remain in that layer. Preferably some further penetrates to the dermis and some cannabinoid penetrates further into the hypodermal layer, to be absorbed systemically. The skin to which the composition is delivered is preferably mammalian skin, more preferably human mammalian skin.

The compositions of the invention may further contain (i) further volatile solvents such as low molecular weight alcohols, and/or (ii) less volatile solvents such as fatty alcohols and/or alkyl polypropylene glycol/polyethylene glycol ethers (alkyl PEG/PPG ethers). The less volatile solvent is called the residual solvent as it may remain on the skin after evaporation of the siloxane (and the further volatile solvent if it is present) These additional volatile and residual solvent excipients may further enhance the capacity of the compositions of the invention to produce concentrated cannabinoid solutions in situ, and/or facilitate the delivery of the cannabinoid to the epidermis and the dermis for the treatment of inflammatory skin conditions.

In accordance with the present invention, there is provided a method for treating or preventing an inflammatory skin condition in a patient in need of such treatment, the method comprising topically administering a prophylactically or therapeutically effective amount of pharmaceutical composition according to the invention.

In accordance with the present invention, there is provided a method for use of a cannabinoid and a siloxane for the manufacture of a pharmaceutical composition for the prevention or treatment of an inflammatory skin condition a patient in need thereof.

In accordance with the present invention, there is provided a method for use of a topical composition according to the invention for the prevention or treatment of an inflammatory skin condition.

In one embodiment, the pharmaceutical composition is a topical composition.

### DESCRIPTION OF THE FIGURES

FIG. 1: Graphical representation of the data shown in Table 10 for delivered CBD. Data is shown in μg/cm². A Dixon's Qtest with 95% confidence was first run on the data to identify and remove outliers.

FIG. 2: Graphical representation of the data shown in Table 10 for delivered CBD. Data is shown in μg/cm². A Dixon's Qtest with 95% confidence was first run on the data to identify and remove outliers.

FIG. 3: Graphical representation of the data shown in Table 11 for delivered CBD. Data is shown in percent delivery. A Dixon's Qtest with 95% confidence was first run on the data sets to identify and remove outliers.

FIG. 4: Graphical representation of the data shown in Table 11 for delivered CBD. Data is shown in percent delivery. A Dixon's Qtest with 95% confidence was first run on the data sets to identify and remove outliers.

FIG. 5: Graphical representation of the data shown in Table 12 for delivered CBD. Data is shown in percent delivery. A Dixon's Qtest with 95% confidence was first run on the data sets to identify and remove outliers.

FIG. 6: Graphical representation of data shown in Table 13 for CBD delivered into the skin. Data is shown in μg/g tissue. A Dixon's Qtest with 95% confidence was first run on the data to identify and remove outliers.

### DETAILED DESCRIPTION OF THE INVENTION

### The Endocannabinoid System (ECS), Cannabinoids, Cannabidiol and Inflammatory Skin Conditions

Identification of the main cannabinoid receptors (CB1 and CB2), their endogenous lipid ligands (endocannabinoids), biosynthetic pathways and metabolizing enzymes (collectively termed the ECS), coupled with the discovery and/or rational design of numerous exogenous ligands for CB receptors, has triggered an exponential growth in studies exploring the continuously growing regulatory functions of this newly discovered physiological system both in health and disease.

Modulating the activity of the ECS holds therapeutic potential for a multitude of diseases and pathological conditions affecting humans, ranging from inflammatory, neurodegenerative, gastrointestinal, liver, cardiovascular disorders and obesity, to ischemia/reperfusion injury, cancer and pain.

The most extensively studied endocannabinoids are anandamide (N arachidonoylethanolamine, AEA) and 2-arachidonoylglycerol (2-AG). Multiple pathways are involved in synthesis and cellular uptake of these lipid mediators. The most common degradation pathways for AEA and 2-AG are the fatty acid amid hydrolase (FAAH) and monoacylglycerol lipase (MAGL) enzyme. Endocannabinoids, similar to Δ⁹-tetrahydrocannabinol (THC; the main active ingredient of the plant Cannabis sativa), predominantly exert their physiological effects via two main G-protein-coupled cannabinoid receptors; however, numerous additional signalling mechanisms and receptor systems (e.g. transient receptor potential cation channel, subfamily V, member 1; TRPV1) might also be involved. Initially, the CB1-mediated effects were described centrally and CB1 receptors were thought to be restricted to the central nervous system, whereas CB2 was first identified at the periphery in immune cells.

The classical steps of AD pathogenesis are the following:
A skin barrier defect or entry of a skin irritant triggers the release of IL-25, IL-33, and thymic stromal lymphopoietin (TSLP) from keratinocytes, which activate dendritic cells (antigen-presenting cells in the skin) and Langerhans cells.
During the “acute phase” of onset, dendritic cells cause excessive Th2, T-helper 22 (Th22), and T-helper 17 (Th17) cell activation (note that these changes continue into the “chronic phase” of the disease).
   Th2 cells produce IL-4, IL-13, and IL-31, which then induce changes in keratinocyte gene expression, disrupt skin barrier function, and trigger itch symptoms. IL-4 and IL-14 can increase additional TSLP release from keratinocytes, which causes further Th2 cell activation.
   Activated Th22 cells release IL-22 which promotes keratinocyte hyperplasia, downregulates keratinocyte differentiation, and synergizes with IL-17 to induce pro-inflammatory S100 genes.
   Activated Th17 cells release IL-17 which can regulate S100 protein and gene expression.
During the chronic stage (day 3 onward), dendritic cells recruit T-helper 1 (Th1) cell populations via IL-12 and continue to recruit Th22 and Th17 cells. Th1 cells release interferon-γ (IFN-γ), which may decrease the role of Th2 cells in the disease. Th1, Th22, and Th17 cells induce responses that continue to attract additional immune cells to the epidermis, alter keratinocyte differentiation, and induce epidermal thickening.

It is estimated that Th2 cell response is dominant in ˜80% of AD cases (extrinsic AD), but in other instances (intrinsic AD), there is a shift to a more pronounced Th22 and Th17 response. [D'Erme 2017] A recent study suggests that IL-17 may have a more dominant role in AD than proposed in classical models [Tan 2017]:
Compared to healthy children, IL-17 protein levels were elevated in AD skin lesions, but not in the serum of children with AD, indicating that IL-17 acts locally.
The effects of 2,4-dinitrochlorobenzene (DNCB; used to induce a model of AD in mice) were evaluated in IL-17 knockout and wild-type C57Bl/6 mice. DNCB was able to induce AD-like lesions in both types of mice; however, epidermal and dermal thickness of the lesions in the IL-17 knockout mice were significantly decreased compared to what was observed in wild-type mice.
Skin mRNA levels of the Th2 cytokines IL-4 and IL-13 were decreased in IL-17 knockout mice compared to wild-type mice; however, there was no difference in skin mRNA expression levels of IFN-γ. Splenocytes isolated from naïve IL-17 knockout mice released less IL-4 following concanavalin A (ConA) stimulation (a model of T-cell activation) compared to splenocytes from treated wild-type mice.

IL-17 has been shown to trigger a pro-inflammatory response in an immortalized human keratinocyte cell line (HaCaT cells). The addition of IL-17 increased the release of pro-inflammatory IL-6 and IL-8, but not IL-1p. This suggests that IL-17 may play a key role in the immune response associated with AD.

CBD may play a beneficial role in decreasing unwanted skin cell growth and skin inflammation associated with many human inflammatory skin diseases.

It is considered that CBD may:
inhibit hyperproliferation of keratinocytes;
exert universal anti-inflammatory actions such as:
   decrease primed T-cell activity and also inhibit subsequent B-cell response;
   suppress multiple T-cell populations and inhibit general T-cell activation;
   decrease concentrations of pro-inflammatory mediators and also increase the release of anti-inflammatory cytokines;
   inhibit the effects of IFN-γ and/or decrease IFN-γ levels;
   inhibit the migration, proliferation and cell maturation processes involved in Th17, Th1, and Th2 immune responses; and
have direct antioxidant effects.

Without being held to any theory, we believe that the mode of action of CBD for inflammatory skin diseases involves the suppression of mediators of inflammatory responses. There is a physiological regulatory function of the endocannabinoid system (ECS) in proliferation, differentiation, apoptosis and cytokine, mediator and hormone production of various cell types of the skin and appendages (e.g. hair follicle, sebaceous gland).

In vitro studies have shown CBD to stimulate the human vanilloid receptor type 1 (VR1) using HEK-hVR1 transfected cells with a maximum effect similar in efficacy to that of capsaicin, and to inhibit anandamide (an endogenous CBD neurotransmitter) using rat basophilic leukemia cells [Bisogno 2001, Mechoulam 2002]. These findings have suggested a mode of action for the anti-inflammatory properties of CBD. In vivo studies with intravenous (i.v.) administration of CBD (1 mg/kg) attenuated ovalbumin-induced airway obstruction in sensitized guinea-pigs, indicating a potential role of CBD in reducing immune-induced inflammatory reactions [DudasovA 2013]. Similarly, CBD (5 mg/kg, i.v.) given to rats once daily for 4 weeks attenuated cardiac inflammation produced by doxorubicin [Fouada 2013].

Unfortunately, due to its highly lipophilic nature, cannabinoids such as cannabidiol are poorly absorbed through membranes such as the skin. Therefore, the success of administering therapeutically effective quantities of a cannabinoid such as cannabidiol to a mammal in need thereof within a reasonable time frame and over a suitable surface area has been substantially limited.

### Composition

The present invention is based on the surprising discovery that a cannabinoid can be dissolved in a siloxane to form a pharmaceutical composition. Optionally, the cannabinoid is cannabidiol. The pharmaceutical composition may be topically applied, after which at least some of the siloxane evaporates to concentrate the cannabinoid in situ, facilitating permeation to the therapeutically relevant regions of the skin (preferably the epidermis and dermal layer) for the treatment of inflammatory skin conditions.

There is therefore provided a pharmaceutical composition comprising a cannabinoid and a siloxane wherein the cannabinoid is dissolved in the composition. In accordance with one embodiment, the cannabinoid is cannabidiol. In accordance with another aspect of the invention, the pharmaceutical composition is a topical pharmaceutical composition. The siloxane forms a volatile solvent for the cannabinoid.

Inflammatory skin conditions are the most common problem in dermatology. They come in many forms, from occasional rashes accompanied by itching and redness to chronic conditions such as dermatitis (eczema), rosacea, seborrheic dermatitis, and psoriasis. However, they are all linked by one common factor, inflammation. It has been found that the inflammatory markers (cytokines) produced by skin and immune cells that are required for the development of an inflammatory response, such as atopic dermatitis and radiation dermatitis. The present invention comprises active agents, in the form of cannabinoids, that suppress the production of a variety of inflammatory responses in cultured skin cells (keratinocytes and fibroblasts), and immune cells (monocytes and T-lymphocytes) and in intact living skin. As a result of blocking these inflammatory processes in the skin, the present compounds in the form of cannabinoids are able to effectively reduce or eliminate a variety of inflammatory symptoms that occur with common skin problem (see Kupczyk et al (2009) Cannabinoid system in the skin—a possible target for future therapies in dermatology Exp Dermatol. 18(8):669-79

High concentrations of dissolved cannabinoids, including cannabidiol (as opposed to solid cannabinoids) are expected to be advantageous in terms of enhancing the relevant extent of delivery into the skin, particularly the epidermis (including the epidermal basal layer), with some penetration into the dermis. It is thought that the high concentration of dissolved cannabinoids on the outer surface of the skin causes a concentration gradient that enhances penetration of the cannabinoid into the skin, particularly the epidermis and the dermis.

In order to achieve local distribution for the treatment of an inflammatory skin condition, it is advantageous for the majority of the cannabinoid, such as cannabidiol, to penetrate into the epidermis and preferably remain there, and for some cannabinoid to further penetrate to the dermis and the hypodermal layer, to be absorbed systemically. In such a case, the cannabidiol would concentrate mainly in the epidermis, thus maximizing its local effect. Not only does the localized effect increase the potential therapeutic benefit, it potentially lessens the frequency and severity of any potential side-effects associated with systemic cannabinoid administration, because the amount of active compound circulating in the patient is reduced.

In one preferred embodiment, the composition is non-aqueous. In another preferred embodiment, the composition does not comprise a preservative.

The present invention is based at least in part on the surprising discovery that cannabinoids can be topically administered as (i) concentrated solutions of cannabinoid in siloxane, or (ii) suspensions of crystalline cannabinoids in concentrated solutions of cannabinoid in siloxane. In either case, the preferred cannabinoid is cannabinol. The compositions of the present invention may form a highly concentrated, non-crystalline, thin layer of a cannabinoid on the skin surface, after partial or complete evaporation of the volatile siloxane, and without crystallization of the cannabinoid.

By using the volatile solvent siloxane, one can achieve much higher, non-crystalline (i.e., in solution), concentrations of cannabinoids. The cannabinoids can be dissolved in much higher concentrations of the volatile solvent siloxane than many other less volatile solvents, and then once applied to the skin and the volatile siloxane has evaporated, the cannabinoids remain on the skin in high concentrations.

The cannabinoids are preferably kept in a non-crystalline form on the skin after evaporation of the siloxane by the addition of a less volatile solvent than siloxane. This less volatile solvent is called the residual solvent, as it preferably remains on the skin after evaporation of the volatile solvent (siloxane and optionally another volatile solvent such as a low molecular weight alcohol) to keep the cannabinoid in a non-crystalline state after evaporation of the siloxane. Preferably the residual solvent is an alkyl polypropylene glycol/polyethylene glycol ether and/or a fatty acid alcohol. Preferably the residual solvent has a low volatility such that less than 5% would evaporate at skin temperature over 24 hours. Preferably, the residual solvent has a chain structure that has a hydrophobic end and a hydrophilic end. Preferably the residual solvent is a liquid at or below 32° C. Preferably the residual solvent dissolves siloxane. Preferably the residual solvent maintains the cannabinoid in non-crystalline form in concentrations of 20% up to 70% cannabinoid.

The total amount of the volatile solvent (siloxane and optionally another volatile solvent such as a low molecular weight alcohol), and the residual solvent if present, required is sufficient to keep the cannabinoid non-crystalline at room temperature for between about 2-8 hours once the composition is applied to the skin.

TABLE 1
Example concentration of CBD on skin after
evaporation of volatile solvents

| | | | | Final CBD concen- |
|---|---|---|---|---|
| | Initial | | | tration in residual |
| | CBD | | | solvent(s) after |
| | Concen- | Volatile | Residual | evaporation of |
| Formula- | tration | Component(s) | solvent(s) | volatile component(s) |
| tion | % w/w | % w/w | % w/w | % w/w |
| 1 | 0.1 | 99.7 | 0.2 | 33.3 |
| 2 | 0.5 | 99.3 | 0.2 | 71.4 |
| 3 | 1.0 | 98.8 | 0.2 | 83.3 |
| 4 | 1.0 | 98.0 | 1.0 | 50.0 |
| 5 | 5.0 | 94.0 | 1.0 | 83.3 |
| 6 | 10.0 | 89.0 | 1.0 | 90.9 |
| 7 | 1.0 | 97.0 | 2.0 | 33.3 |
| 8 | 5.0 | 93.0 | 2.0 | 71.4 |
| 9 | 10.0 | 88.0 | 2.0 | 83.3 |
| 10 | 1.0 | 96.0 | 3.0 | 25.0 |
| 11 | 5.0 | 92.0 | 3.0 | 60.0 |
| 12 | 10.0 | 87.0 | 3.0 | 76.9 |

Such administration is expected to result in enhanced delivery of a cannabinoid, such as cannabidiol, to the epidermis and dermis of the skin, which is expected to be effective in significantly reducing, and therefore, treating an inflammatory skin condition in patients in need of such treatment.

In addition to enhanced delivery, the present invention may allow larger doses of cannabinoids, such as cannabidiol, to be applied without having to have a thick layer of residue that would be rubbed off or be unacceptable to the user. The topical pharmaceutical compositions of the present invention allow more rapid delivery of the cannabinoid due to the metastable high driving force or supersaturation of the composition. In summary, it is thought that the high concentration of dissolved cannabinoids on the outer surface of the skin causes a concentration gradient that enhances penetration of the cannabinoid into the epidermis and dermis.

Therefore, in one aspect, the present invention comprises a topical composition comprising a solution of a cannabinoid in a siloxane. In one embodiment, the cannabinoid is cannabidiol.

Definitions: CBD: cannabidiol (CPD), IPA: isopropyl alcohol, MO: occlusive mineral oil (a viscous liquid petrolatum), HDS: hexylmethyldisiloxane, PMS: polymethylsiloxane 10⁶ cSt, HDA: 2-hexyldecyl alcohol, PG: propylene glycol, OA: oleyl alcohol, EtOH: ethanol, ODDA: octyldodecyl alcohol, AE: arlamol E, and Klucel MF: hydroxypropylcellulose (brand name Klucel® MF from Ashland, Inc.).

The preferred ratio of cannabinoid to siloxane to residual solvent is selected from the range consisting of (w/w %): 0.5-20% cannabinoid, between 1-99% siloxane and between 0.1-99% residual solvent; between 5-20% cannabinoid, between 4-70% siloxane and between 1%-70% residual solvent; between 1-15% cannabinoid, between 20-95% siloxane and between 1-15% residual solvent.

In one preferred embodiment, the composition is selected from the group consisting of (w/w %):
5% CBD/10% OA/10% PG/10% HDS/65% IPA
14% CBD/9% OA/9% PG/9% HDS/59% IPA
14% CBD/4.5% OA/13.5% PG/4.5% HDS/63.5% IPA
15% CBD/5% PMS/10% OA/70% HDS
15% CBD/10% argan oil/10% HDS/65% IPA
10% CBD/7% argan oil/7% ISA/9% PMS/67% HDS
15% CBD/13% IPA/7% PMS/66% HDS
15% CBD/12.5% HDA/6% PMS/66.5% HDS
15% CBD/12.5% ODDA/6% PMS/66.5% HDS
15% CBD/10% HDA/40% IPA/35% HDS
15% CBD/10% ODDA/40% IPA/35% HDS
7.2% CBD/6.3% PMS/1.4% MO/1.8% IPA/83.3% HDS
20% CBD/10% ODDA/70% IPA
9.5 CBD/4.8% ODDA/57.1% EtOH/28.6% HDS
10% CBD/12.5% PMS/4.5% IPA/72% HDS
5% CBD/2.5% HDA/50% IPA/41% HDS/1% KlucelMF
5% CBD/3.33% HDA/50% IPA/40.67% HDS/1% KlucelMF
5% CBD/3.33% HDA/75% IPA/15.67% HDS/1% KlucelMF
10% CBD/6.67% HDA/75% IPA/7.33% HDS/1% KlucelMF
15% CBD/10% HDA/70% IPA/4% HDS/1% KlucelMF
15% CBD/7.5% HDA/70% IPA/6% HDS/1.5% KlucelMF
5% CBD/2.5% HDA/1% PMS/91.5% HDS
10% CBD/5% HDA/1% PMS/84% HDS
15% CBD/7.5% HDA/1% PMS/1% IPA/1% D5/74.5% HDS
5% CBD/2% AE/1% PMS/92% HDS
10% CBD/4% AE/1% PMS/1% IPA/84% HDS
5% CBD/2.5% HDA/1% PMS/91.5% HDS
5% CBD/1.7% HDA/1.2% PMS/92.1% HDS
5.25% CBD/1.15% PMS/1.22% IPA/92.38% HDS
5% CBD/2.5% AE/1% PMS/91.5% HDS
5% CBD/1% AE/1% PMS/93% HDS
5% CBD/2.5% IPM/1% PMS/1% IPA/90.5% HDS
10% CBD/4% AE/1% PMS/1% IPA/84% HDS
5% CBD/2% AE/1% PMS/92% HDS
5% CBD/2.5% HDA/5% PMS/87.5% HDS
10% CBD/6.67% HDA/5% PMS/78.33% HDS
15% CBD/7.5% HDA/5% PMS/1% IPA/71.5% HDS
15% CBD/7.5% HDA/10% PMS/1% IPA/66.5% HDS

In a further preferred embodiment, the composition is selected from the group consisting of:
5% CBD/3.33% HDA/50% IPA/40.67% HDS/1% KlucelMF
5% CBD/3.33% HDA/75% IPA/15.67% HDS/1% KlucelMF
10% CBD/6.67% HDA/75% IPA/7.33% HDS/1% KlucelMF
15% CBD/10% HDA/70% IPA/4% HDS/1% KlucelMF
15% CBD/7.5% HDA/70% IPA/6% HDS/1.5% KlucelMF
5% CBD/2% AE/1% PMS/92% HDS
10% CBD/4% AE/1% PMS/1% IPA/84% HDS
5% CBD/2.5% HDA/1% PMS/91.5% HDS
10% CBD/5% HDA/1% PMS/84% HDS
15% CBD/7.5% HDA/1% PMS/1% IPA/1% D5/74.5% HDS
5% CBD/1.7% HDA/1.2% PMS/92.1% HDS
5.25% CBD/1.15% PMS/1.22% IPA/92.38% HDS

In one preferred embodiment, the following formulations are solutions: 5% CBD/10% OA/10% PG/10% HDS/65% IPA, 14% CBD/9% OA/9% PG/9% HDS/59% IPA, 14% CBD/4.5% OA/13.5% PG/4.5% HDS/63.5% IPA, and 5% CBD/2% AE/1% PMS/92% HDS. In another preferred embodiment, these formulations are gelled with 1% Klucel.

In one preferred form, the composition is a gel. In another preferred form, the composition is a spray. The composition may or may not contain water. Preferably, the composition does not contain water, i.e. it is non-aqueous.

### Siloxane

Siloxanes do not burn, sting or have an odour, and thus are highly advantageous for topical application for the treatment of an inflammatory skin condition. Importantly for the compositions of the present invention, siloxanes due to their low molecular weight, are highly volatile.

In one embodiment, the siloxane contains two or three silicon atoms. The siloxanes may have between one and eight methyl groups. In one embodiment, the siloxane is selected from the group consisting of: hexamethyldisiloxane, octamethyltrisiloxane and combinations thereof. These are the most volatile siloxanes, and are thus the most advantageous. Preferably the level of volatility of the siloxane is about the same as that of isopropyl alcohol.

In another embodiment, the siloxane contains 4 or 5 silicon atoms, and is, for example, decamethyltetrasiloxane or dodecamethylpentasiloxane. In another embodiment, the siloxane is a cyclical 4 or 5 silicon atom compound such octamethylcyclotetrasiloxane (CAS #556-67-2) or decamethylcyclopentasiloxane (CAS #541-02-6).

In certain embodiments, further improvements in the solubility and crystallinity characteristics of the cannabinoid in the siloxane may be achieved by the addition of a further volatile solvent in the form of an alcohol, including a low molecular weight alcohol. An improvement in the solubility and crystallinity characteristics of the cannabinoid in the siloxane may also be achieved by the addition of an alkyl PEG/PPG ether and/or a fatty alcohol.

### Alkyl Polypropylene Glycol/Polyethylene Glycol Ethers

In certain embodiments, further improvements in the solubility characteristics of the cannabinoid, such as cannabidiol, in the siloxane may be achieved by the addition of alkyl polypropylene glycol/polyethylene glycol ethers (alkyl PEG/PPG ethers). The properties of alkyl PEG/PPG ethers, as well as suitable alkyl PEG/PPG ethers that can be used in accordance with this invention, are discussed in the Cosmetic Ingredient Review (CIR) Expert Panel 2013 “Safety Assessment of Alkyl PEG/PPG Ethers as Used in Cosmetics” Report (www.cir-safety.org/sites/default/files/PEGPPG062013tent.pdf: accessed 21 Dec. 2016) and the contents of that document are incorporated herein.

The alkyl PEG/PPG ethers also act as a residual solvent to assist in maintaining the cannabinoid in a non-crystalline state after evaporation of some or all of the siloxane and the optional low molecular weight alcohol.

Advantageously, in some embodiments, the composition also comprises one or more alkyl PEG/PPG ethers. Alkyl PEG/PPG ethers are the reaction products of an alkyl alcohol and one or more equivalents each of ethylene oxide and propylene oxide (forming repeats of polyethylene glycol (PEG) and polypropylene glycol (PPG), respectively).

The inventors have found that the addition of alkyl PEG/PPG ethers, including polypropylene glycol ethers of stearyl alcohol and butyl alcohol, can improve the solubility of cannabinoids, such as cannabidiol, in siloxane solvents. This ability to increase the concentration of the cannabinoid in the initial composition and in the final composition on the skin after application and evaporation makes it possible to achieve high residual concentrations of cannabinoids on the skin. The alkyl PEG/PPG ethers provide a residual solvent that can retain the cannabinoid in solution at an exceptionally high concentration after evaporation of the volatile solvent or solvent mixture.

Advantageously, in some embodiments, the alkyl PEG/PPG ethers are liquids at ambient temperatures. Preferably the alkyl PEG/PPG ethers are liquids at about 30° C., or less, or at about 25° C.

Advantageously, in some embodiments, the alkyl PEG/PPG ethers have a low volatility such that less than 5% would evaporate at skin temperature over 24 hours.

Advantageously, in some embodiments, the alkyl PEG/PPG ether has a PEG/PPG chain length of between 10-50 PG units and an ether component of between 2-20 carbons, wherein the sum of the PG units and the carbons of the ether component is preferably between 20 and 60. A range of alkyl PEG/PPG ethers are discussed in the Cosmetic Ingredient Review (CIR) Expert Panel 2013 “Safety Assessment of Alkyl PEG/PPG Ethers as Used in Cosmetics” Report (www.cir-safety.org/sites/default/files/PEGPPG062013tent.pdf; accessed 21 Dec. 2016) and the contents of that document, including the lists of alkyl PEG/PPG ethers, are incorporated herein.

Advantageously, in some embodiments, the alkyl PEG/PPG ether is selected from the group consisting of: polypropylene glycol ethers of stearyl alcohol or butyl alcohol and combinations thereof.

In specific embodiments, the alkyl PEG/PPG stearyl ether or butyl ether is selected from the group consisting of: polypropylene glycol (PPG) stearyl ethers and polypropylene glycol butyl ethers such as PPG-15 stearyl ether and PPG-40 butyl ether and combinations thereof.

In specific embodiments, the relative amount of alkyl PEG/PPG ether is selected from the following group; at least 1% w/w, at least 2% w/w, at least 3% w/w, at least 4% w/w, at least 5% w/w. In specific embodiments, the maximum concentration of the alkyl PEG/PPG ether is 50% w/w. In specific embodiments, the maximum concentration of the alkyl PEG/PPG ether is 80% w/w.

Preferably the amount of alkyl PEG/PPG ether is sufficient to keep the cannabinoid is a non-crystalline form on the skin after partial or complete evaporation of the more volatile solvent or solvents.

### Low Molecular Weight Alcohol

Advantageously, in some embodiments, the topical composition also comprises a low molecular weight alcohol. The inventors have found that small amounts of a low molecular weight alcohol may improve the solubility of cannabinoids, such as cannabidiol, in siloxane solvents. This ability to increase the concentration of the cannabinoid in the initial composition makes it possible to achieve high residual concentrations of cannabinoids on the skin after application. Preferably the low molecular weight alcohol forms a further volatile solvent in addition to the siloxane. Preferably the level of volatility of the low molecular weight alcohol is about the same as that of isopropyl alcohol. The addition of a further volatile solvent such as a low molecular weight alcohol may be of particular advantage if the concentration of cannabinoid in the initial composition is very high.

Advantageously, in some embodiments, the low molecular weight alcohol is a liquid at ambient temperatures. Preferably the low molecular weight alcohol is liquid at about 30° C., or less, or at about 25° C. Preferably the level of volatility of the low molecular weight alcohol is about the same as that of isopropyl alcohol.

Advantageously, in some embodiments, the low molecular weight alcohol is selected from the group consisting of: C₂₋₆ alcohols, and combinations thereof. Advantageously, in some embodiments, the low molecular weight alcohol is selected from the group consisting of: C₂₋₄ alcohols, and combinations thereof.

In specific embodiments, the low molecular weight alcohol is selected from the group consisting of: ethyl alcohol (or ethanol), n-propanol, isopropyl alcohol, butanol and combinations thereof.

In specific embodiments, the relative amount of low molecular weight alcohol selected from the following group: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w. In specific embodiments, the maximum concentration of the low molecular weight alcohol is 50% w/w. In specific embodiments, the maximum concentration of the low molecular weight alcohol is 60% w/w, 70% w/w, 80% w/w. The amount of low molecular weight alcohol may be between 1% w/w and 50% w/w, 1% w/w and 40%, 1% w/w and 30% w/w, 1% w/w and 20% w/w, 1% w/w and 10% w/w.

### Fatty Alcohol

Advantageously, in certain embodiments, the topical composition is further characterised in that the composition comprises a fatty alcohol. The purpose of the fatty alcohol is to act as a solvent for the cannabinoid once the volatile components, such as the siloxane and, optionally, the low molecular weight alcohol, have evaporated. In specific embodiments the fatty alcohol is a C₁₂₋₂₂ fatty alcohol. In specific embodiments, the fatty alcohol is a C₁₆₋₂₂ fatty alcohol. In specific embodiments, the fatty alcohol is selected from the group consisting of: oleyl alcohol, isostearyl alcohol, octyldodecyl alcohol, 2-hexyl decyl alcohol.

In specific embodiments, the relative amount of fatty alcohol selected from the following group; at least 2% w/w, at least 3% w/w, at least 4% w/w, at least 5% w/w. In specific embodiments, the maximum concentration of the fatty alcohol is 50% w/w. In specific embodiments, the maximum concentration of the fatty alcohol is 80% w/w.

Preferably the amount of fatty alcohol is sufficient to keep the cannabinoid is a non-crystalline form on the skin after partial or complete evaporation of the more volatile solvent or solvents.

### Cannabinoid

Preferably, the cannabinoid is cannabinol. Alternatively, the cannabinoid is any compound that interacts with the cannabinoid receptor. This may include various cannabinoid mimetics, such as certain tetrahydropyran analogs (e.g., Δ9-tetrahydrocannabinol, Δ8-tetrahydro-cannabinol, 6,6,9-trimethyl-3-pentyl-6H-dibenzo [b,d]pyran-1-ol, 3-(1, 1-dimethylheptyl)-6, 6a, 7, 8, 10, 10a-hexahydro-1-hydroxy-6,6-dimethyl-9H-dibenzo[b,d]pyran-9-one, (−) -(3S,4S)-7-hydroxy-Δ6-tetrahydrocannabinol-1,1-dimethylheptyl,(+)-(3S,4S)-7-hydroxy-Δ6-tetrahydrocannabinol-1,1-dimethylheptyl, 11-hydroxy-Δ9-tetrahydrocannabinol, and Δ8-tetrahydrocannabinol-11-oic acid)); certain piperidine analogs (e.g., (−)-(6S,6aR,9R, 10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl); cannabinol; cannbigerol; tetrahydrocannabivarin; cammabidvarin; cannabichromene; and includes synthetic cannabinoids (such as nabilone, rimonabant, JWH-018, JWH-073, CP-55940, dimethylheptlpryan, HU-210, HU-331, SR144528, WIN 55,212-2, JWH-133, Levonantradol, and AM-2201) as well as salts and analogs thereof.

In certain embodiments, the concentration of cannabinoid in the topical composition of the invention may be selected from the group consisting of: at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, and at least 15% w/w.

In certain embodiments, the concentration of cannabinoid in the topical composition may be selected from the group consisting of: at least 20% w/w, at least 30% w/w at least 40% w/w, at least 50% w/w, at least 60% w/w, at least 65% w/w, at least 70% w/w, at least 80% w/w, at least 90% w/w, at least 95% w/w and at least 99% w/w. Such concentrations may be achieved after at least partial evaporation of the volatile siloxane and, optionally, low molecular weight alcohol components.

In certain embodiments, the concentration of cannabinoid in the topical composition may be within a range with a lower limit selected from the group consisting of: 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, and 15% w/w;

and an upper limit selected from the group consisting of:
20% w/w, 30% w/w, 40% w/w, 50% w/w, 60% w/w, 65% w/w, 70% w/w, 80% w/w, 90% w/w, 95% w/w, and 99% w/w.

In certain embodiments, the concentration of the cannabinoid in the topical composition may be within a range selected from the group consisting of:

1% w/w, 2% w/w to 99% w/w, 3% w/w to 70% w/w, 4% w/w to 70% w/w, 5% w/w to 70% w/w, 6% w/w to 70% w/w, 7% w/w to 70% w/w, 8% w/w to 99% w/w, 9% w/w to 99% w/w, 10% w/w to 99% w/w, 11% w/w to 99% w/w, 12% w/w to 99% w/w, 13% w/w to 99% w/w, 14% w/w to 99% w/w, and 15% w/w to 99% w/w.

In certain embodiments, the concentration of the cannabinoid in the topical composition may be within a range selected from the group consisting of:

1% w/w, 2% w/w to 95% w/w, 3% w/w to 95% w/w, 4% w/w to 95% w/w, 5% w/w to 95% w/w, 6% w/w to 95% w/w, 7% w/w to 95% w/w, 8% w/w to 95% w/w, 9% w/w to 95% w/w, 10% w/w to 95% w/w, 11% w/w to 95% w/w, 12% w/w to 95% w/w, 13% w/w to 95% w/w, 14% w/w to 95% w/w, and 15% w/w to 95% w/w.

In certain embodiments, the concentration of the cannabinoid in the topical composition may be within a range selected from the group consisting of:

1% w/w, 2% w/w to 90% w/w, 3% w/w to 90% w/w, 4% w/w to 90% w/w, 5% w/w to 90% w/w, 6% w/w to 90% w/w, 7% w/w to 90% w/w, 8% w/w to 90% w/w, 9% w/w to 90% w/w, 10% w/w to 90% w/w, 11% w/w to 90% w/w, 12% w/w to 90% w/w, 13% w/w to 90% w/w, 14% w/w to 90% w/w, and 15% w/w to 90% w/w.

In certain embodiments, the concentration of the cannabinoid in the topical composition may be within a range selected from the group consisting of:

1% w/w, 2% w/w to 80% w/w, 3% w/w to 80% w/w, 4% w/w to 80% w/w, 5% w/w to 80% w/w, 6% w/w to 80% w/w, 7% w/w to 80% w/w, 8% w/w to 80% w/w, 9% w/w to 80% w/w, 10% w/w to 80% w/w, 11% w/w to 80% w/w, 12% w/w to 80% w/w, 13% w/w to 80% w/w, 14% w/w to 80% w/w, and 15% w/w to 80% w/w.

In certain embodiments, the concentration of the cannabinoid in the topical composition may be within a range selected from the group consisting of:

1% w/w, 2% w/w to 70% w/w, 3% w/w to 70% w/w, 4% w/w to 70% w/w, 5% w/w to 70% w/w, 6% w/w to 70% w/w, 7% w/w to 70% w/w, 8% w/w to 70% w/w, 9% w/w to 70% w/w, 10% w/w to 70% w/w, 11% w/w to 70% w/w, 12% w/w to 70% w/w, 13% w/w to 70% w/w, 14% w/w to 70% w/w, and 15% w/w to 70% w/w.

In certain embodiments, the concentration of the cannabinoid in the topical composition may be within a range selected from the group consisting of:

1% w/w, 2% w/w to 65% w/w, 3% w/w to 65% w/w, 4% w/w to 65% w/w, 5% w/w to 65% w/w, 6% w/w to 65% w/w, 7% w/w to 65% w/w, 8% w/w to 65% w/w, 9% w/w to 65% w/w, 10% w/w to 65% w/w, 11% w/w to 65% w/w, 12% w/w to 65% w/w, 13% w/w to 65% w/w, 14% w/w to 65% w/w, and 15% w/w to 65% w/w.

In certain embodiments, the concentration of the cannabinoid in the topical composition may be within a range selected from the group consisting of:

1% w/w, 2% w/w to 60% w/w, 3% w/w to 60% w/w, 4% w/w to 60% w/w, 5% w/w to 60% w/w, 6% w/w to 60% w/w, 7% w/w to 60% w/w, 8% w/w to 60% w/w, 9% w/w to 60% w/w, 10% w/w to 60% w/w, 11% w/w to 60% w/w, 12% w/w to 60% w/w, 13% w/w to 60% w/w, 14% w/w to 60% w/w, and 15% w/w to 60% w/w.

In certain embodiments, the concentration of the cannabinoid in the topical composition may be within a range selected from the group consisting of:

1% w/w, 2% w/w to 50% w/w, 3% w/w to 50% w/w, 4% w/w to 50% w/w, 5% w/w to 50% w/w, 6% w/w to 50% w/w, 7% w/w to 50% w/w, 8% w/w to 50% w/w, 9% w/w to 50% w/w, 10% w/w to 50% w/w, 11% w/w to 50% w/w, 12% w/w to 50% w/w, 13% w/w to 50% w/w, 14% w/w to 50% w/w, and 15% w/w to 50% w/w.

In certain embodiments, the concentration of the cannabinoid in the topical composition may be within a range selected from the group consisting of:

1% w/w, 2% w/w to 40% w/w, 3% w/w to 40% w/w, 4% w/w to 40% w/w, 5% w/w to 40% w/w, 6% w/w to 40% w/w, 7% w/w to 40% w/w, 8% w/w to 40% w/w, 9% w/w to 40% w/w, 10% w/w to 40% w/w, 11% w/w to 40% w/w, 12% w/w to 40% w/w, 13% w/w to 40% w/w, 14% w/w to 40% w/w, and 15% w/w to 40% w/w.

In certain embodiments, the concentration of the cannabinoid in the topical composition may be within a range selected from the group consisting of:

1% w/w, 2% w/w to 30% w/w, 3% w/w to 30% w/w, 4% w/w to 30% w/w, 5% w/w to 30% w/w, 6% w/w to 30% w/w, 7% w/w to 30% w/w, 8% w/w to 30% w/w, 9% w/w to 30% w/w, 10% w/w to 30% w/w, 11% w/w to 30% w/w, 12% w/w to 30% w/w, 13% w/w to 30% w/w, 14% w/w to 30% w/w, and 15% w/w to 30% w/w.

In certain embodiments, the concentration of the cannabinoid in the topical composition may be within a range selected from the group consisting of:

1% w/w, 2% w/w to 20% w/w, 3% w/w to 20% w/w, 4% w/w to 20% w/w, 5% w/w to 20% w/w, 6% w/w to 20% w/w, 7% w/w to 20% w/w, 8% w/w to 20% w/w, 9% w/w to 20% w/w, 10% w/w to 20% w/w, 11% w/w to 20% w/w, 12% w/w to 20% w/w, 13% w/w to 20% w/w, 14% w/w to 20% w/w, and 15% w/w to 20% w/w.

### Other Agents

The cannabinoid could be incorporated into a composition with an additional active moiety that is capable of improving the appearance and/or hydration of the skin.

In addition, the composition of the present invention can be used in conjunction with other topically applied analgesic and/or systemically available agents for the treatment of inflammatory skin conditions.

Examples of such analgesic agents include, but are not limited to: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds contemplated by as suitable in the present invention include, but are not limited to morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

Examples of systemically available agents which may be used in conjunction with the present compositions for the treatment of inflammatory skin conditions include, but are not limited to: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

In addition, other active agents may be included in the composition of the present invention, e.g., topically-effective anaesthetics such as xylocaine, cocaine, lidocaine, benzocaine, etc., which may provide a more immediate, if less effective in the long run, level of pain relief until the analgesic agent becomes fully effective.

Still other agents can also be administered, preferably topically, to potentiate the effects of the topically-administered cannabidiol. For example, dextromethorphan, a non-addictive opioid compound, can be co-administered, preferably topically, although parenteral administration is also effective, to enhance the effectiveness of the topically administered agent. Without wishing to be bound by theory, it is believed that dextromethorphan has previously unappreciated analgesic properties in peripheral nerves. Suitable concentrations of dextromethorphan are routinely ascertainable by the skilled worker, and include the normal therapeutic amounts administered parenterally for conventional purposes, e.g., as a cough suppressant, or less, and routinely determinable amounts for topical administration; for example, 1 g of dextromethorphan can be added to a composition disclosed herein to provide additional treatment for inflammatory skin conditions.

In one embodiment, the pharmaceutical composition of the present invention further comprises one or more of the following agents for the treatment of an inflammatory skin condition: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

### Inflammatory Skin Condition Treatment and Therapy

In certain embodiments the topical application of cannabinoid, such as cannabidiol, by way of the compositions of the present invention is expected to reduce the incidence and/or severity of the inflammatory skin condition. Therapeutic effects of the present invention include, but are not limited to, reduction in redness, itch, pain or irritation, a reduction in pimples, papules, blisters or pustules, a reduction in infection, a reduction in dryness, cracking and wrinkling, a reduction of swelling, cracking, weeping, crusting, and scaling and/or a general decrease in inflammation.

In certain embodiments, the topical application of cannabinoid, such as cannabidiol, by way of the compositions of the present invention is expected to improve the symptoms of the inflammatory skin condition.

The term “improve” is used to convey that the present invention changes either the appearance, form, characteristics and/or the physical attributes of the tissue to which it is being provided, applied or administered. The change in form may be demonstrated by any of the following alone or in combination: enhanced appearance of the skin; decreased inflammation of the skin, prevention of inflammation or blisters, decreased spread of blisters, decreased ulceration of the skin, decreased redness, reduction of scarring, reduction in lesions, healing of blisters, reduced skin thickening, closure of wounds and lesions, a reduction in symptoms including, but not limited to, pain, inflammation, itching, milia or other symptoms associated with inflammatory conditions or the like.

A primary advantage of the present invention is expected to be the improvement in the condition of the skin without the typical side effects of conventional therapies. The potential for the present invention is widespread, and the topical application of cannabinoids shows promise as an exciting new method of inflammatory skin condition treatment.

It is expected that treatment of the inflammatory skin condition n accordance with embodiments of the present invention results in improved healing of the skin. For example, when used in the treatment of dermatitis, swollen, cracked or scaled skin is which is treated is expected to heal more quickly and/or completely, compared to when left untreated.

When administered in accordance with the present invention, treatment is expected to result in one or more therapeutic effects. Therapeutic effects in the affected area include, but are not limited to, reduction in redness, itch, pain or irritation, a reduction in pimples, papules, blisters or pustules, a reduction in infection, a reduction in dryness, cracking and wrinkling, less breakdown and loss of collagen and elastin in the skin, a reduction of swelling, cracking, weeping, crusting, and scaling and/or a general decrease in inflammation. One or more of these therapeutic effects are expected to be observed when treatment in accordance with the present invention is made to any of the suitable conditions.

Unless the context requires otherwise, the phrase “inflammatory skin condition” includes skin diseases and skin disorders, and means conditions that are accompanied by a series of clinical signs and symptoms, such as itch, oedema, erythema and abrasion and are induced by various stimulative factors that cause a series of inflammatory reactions in the skin. In some aspects, the inflammatory skin condition may be characterized by ulceration, inflammation, or blistering of the skin. In some embodiments, the inflammatory skin condition may be characterized by a genetic component, an autoimmune component, a circulatory component or combinations thereof. In the present application, the term “inflammatory skin condition” is used interchangeably with “inflammatory skin disease”.

In one embodiment, the “inflammatory skin condition” is selected from the list: rosacea, dermatitis (including radiation dermatitis, atopic dermatitis, allergic and irritant contact dermatitis, seborrheic dermatitis, statis dermatitis), erythemas (sunburns), actinic keratitis (including actinic cheilitis), scarring, hyperpigmentation, lupus erythematosus, pemphigoid, hives, eczema, lichen planus, acrodermatitis, dermatomyositis, inflammatory skin conditions resulting from skin infections (including tinea pedis and tinea versicolor, shingles, mouth ulcers (including stomatitis, canker sore), nappy rash, erysipelas, impetigo, cutaneous candidiasis), or inflammation resulting from bites and stings (including bee stings, ant bites, wasp stings, tick bites, flea bites, scabies infections).

In one embodiment, the “inflammatory skin condition” is selected from the list: cutaneous porphyria, sclerodema, epidermolysis bulosa, decubitus ulcers, pressure ulcers, diabetic ulcers, venous stasis ulcers, sickle cell ulcers, ulcers caused by burns, urticaria, dermatitis herpetiform, arthritis, gout, alopecia, carcinomas, miliaria, skin infections, post-operative care of incisions, post-operative skin care following any variety of plastic surgery operations, skin care following radiation treatment, care of dry, cracked or aged skin and skin lines as well as other conditions affecting the skin and having an inflammatory component, symptoms thereof, or a combination thereof. Symptoms treated may include pain, inflammation, redness, itching, scarring, skin thickening, milia, or a combination thereof.

In one embodiment, the “inflammatory skin condition” is selected from the list: dermatological pain, dermatological inflammation, bacterial skin infections, fungal skin infections, viral skin infections, parasitic skin infections, skin neoplasia, skin neoplasms, pruritus, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, pediculosis, creeping eruption, pityriasis rosea, pityriasis rubra pilaris, edematous, erythema multiform, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, purpura, moniliasis, candidiasis, baldness, androgenetic alopecia, Behcet's syndrome, cholesteatoma, Dercum disease, ectodermal dysplasia, gustatory sweating, nail patella syndrome, telogen effluvium, Hailey-Hailey disease, chemical or thermal skin burns, scleroderma, aging skin, wrinkles, sun spots, necrotizing fasciitis, necrotizing myositis, gangrene, scarring, and vitiligo.

In a specific embodiment, the requires otherwise, the phrase “inflammatory skin condition” means rosacea, radiation dermatitis, erythemas (sunburns), atopic dermatitis, allergic and irritant contact dermatitis, actinic keratitis, acne, scarring, hyperpigmentation, and seborrheic dermatitis or eczema, or other eczemas, or and alopecia areata.

The present invention further provides a method for treating or preventing an inflammatory skin condition in a patient in need of such treatment, the method comprising topically administering a prophylactically or therapeutically effective amount of a topical composition as described herein.

The present invention further provides the use of a cannabinoid and a siloxane for the manufacture of a topical composition, as described herein, for the prevention or treatment of an inflammatory skin condition in a patient in need thereof.

The present invention further provides the use of a topical composition, as described herein, for the prevention or treatment of an inflammatory skin condition.

In one aspect, the present invention is directed to methods of treating an inflammatory skin condition using topical cannabinoids, including cannabidiol. In accordance with certain embodiments, a topical composition of the invention containing cannabinoids such as cannabidiol, is preferably applied topically to an area which is affected by the inflammatory skin condition. Preferably, the application of cannabinoid in accordance with certain embodiments results in reduction in redness, itch, pain or irritation, a reduction in pimples, papules, blisters or pustules, a reduction in infection, a reduction in dryness, cracking and wrinkling, less breakdown and loss of collagen and elastin in the skin, a reduction of swelling, cracking, weeping, crusting, and scaling and/or a general decrease in inflammation.

### Pharmaceutical Composition

Certain embodiments of the present invention comprise any topically acceptable non-transdermally effective carrier vehicle. Preferred topically acceptable vehicles include but are not limited to gels, ointments, and liquids. Administration of the preferred embodiment is performed in accordance with that mode which is most amenable to the topically acceptable form chosen. For example, gels, lotions, creams and ointments are preferably administered by spreading.

The composition may or may not contain water. Preferably, the composition does not contain water, i.e. it is non-aqueous.

The dilution of the cannabinoid in the topical composition can be an important consideration. The cannabinoid concentration in the composition should be high enough that the patient does not need to wait an excessively long time for the composition to dry. On the other hand, the cannabinoid concentration should be dilute enough that a patient can achieve effective coverage of the affected area. Additionally, the composition could include a component which polymerizes in response to exposure to air or ultraviolet radiation.

The amount of composition to be applied will vary depending on the choice of siloxane, low molecular weight alcohol, fatty alcohol, and/or alkyl PEG/PPG ether as well. For example, when the cannabinoid, such as cannabidiol, is administered by spraying a solution of the drug, the total volume in a single dose may be as low as 0.1 ml. When the cannabinoid, such as cannabidiol, is administered in a gel or cream, the total volume may be as high as 3 ml. Conversely, if the inflammatory skin condition comprises scattered lesions, the volume applied to each lesion may be smaller. The carrier selected, and its manner of application, are preferably chosen in consideration of the needs of the patient and the preferences of the administering physician.

In one preferred embodiment, the composition comprises a gel which is preferably administered by spreading the gel onto the affected area. In other preferred embodiments, the composition comprises a liquid, which can be administered by spraying or otherwise applying the liquid onto the affected area.

The quantities of the applied cannabinoid, such as cannabidiol, described herein in the Examples are illustrative only and it is to be appreciated that lesser and greater quantities may be used, which can be routinely optimized by the skilled worker. In general, amounts therapeutically equivalent to 0.1 to 200 mg of cannabinoid, such as cannabidiol, applied to an area of 5-100 cm², are preferred. However, the quantity of cannabinoid used in the topical application of the present invention is typically a small fraction of the typical dosage used in other methods of treatment using these agents, e.g., epilepsy.

In accordance with certain embodiments, the composition is applied to the affected area regularly until relief is obtained. In one preferred embodiment, the composition is administered to the skin of the patient in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention.

In certain embodiments, the composition of the invention may be provided in a form selected from the group comprising, but not limited to a liquid or gel, a leave-on preparation, a wash-off preparation.

In one embodiment, the composition comprises impurities, wherein the quantity of impurities as a percentage of the total weight of the composition is selected from the group consisting of: less than 20% impurities (by total weight of the composition); less than 15% impurities; less than 10% impurities; less than 8% impurities; less than 5% impurities; less than 4% impurities; less than 3% impurities; less than 2% impurities; less than 1% impurities: less than 0.5% impurities; less than 0.1% impurities. In one embodiment, the composition comprises microbial impurities or secondary metabolites, wherein the quantity of microbial impurities as a percentage of the total weight of the composition is selected from the group consisting of: less than 5%; less than 4%; less than 3%; less than 2%; less than 1% s; less than 0.5%; less than 0.1%; less than 0.01%; less than 0.001%. In one embodiment, the composition is sterile and stored in a sealed and sterile container. In one embodiment, the composition contains no detectable level of microbial contamination.

The foregoing embodiments are illustrative of applications in which methods of treating an inflammatory skin condition using a cannabinoid, such as cannabidiol, in accordance with the present invention can be employed. Those of ordinary skill in the art will readily understand that other manners of administration of cannabinoids to treat inflammatory skin conditions are suitable and are in accordance with the present invention as well.

### Definitions

The following definitions in this specification are intended to be interpreted in an illustrative, rather than limiting sense. Therefore, they are to be interpreted inclusively, and are not to be limited to the specific definition recited.

Antagonist: a compound that does not enhance or stimulate the functional properties of a receptor, yet block those actions by an agonist.

Bandage: a dressing used to cover an afflicted area.

Cannabinoid: as used herein, is meant to include compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo [b,d]pyran-1-ol, 3-(1, 1-dimethylheptyl)-6, 6a, 7, 8, 10, 10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl, (+)-(3S,4S)-7-hydroxy-Δ6-tetrahydrocannabinol-1,1-dimethylheptyl, 11-hydroxy-Δ⁹-tetrahydrocannabinol, and A8-tetrahydrocannabinol-11-oic acid)); certain piperidine analogs (e.g., (−)-(6S,6aR,9R, 10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl). Further examples of “cannabinoids” include those compounds described in the references cited below.

Cannabidiol: as used herein, is meant to refer to 2-[3-methyl-6-(1-methylethenyl)-2-cyclohexen-1-yl]-5-pentyl-1,3-benzenediol.

The synthesis of 2-[3-methyl-6-(1-methylethenyl)-2-cyclohexen-1-yl]-5-pentyl-1,3-benzenediol is described, for example, in Petilka et al., Helv. Chim. Acta, 52: 1102 (1969) and in Mechoulam et al., J. Am. Chem. Soc., 87:3273 (1965), which are hereby incorporated by reference.

Central nervous system: the brain and spinal cord.

Dermal: relating to the dermis.

Dressing combine: designed to provide warmth and protection to absorb large quantities of fluid that may drain from an incision or wound; consists of a nonwoven fabric cover enclosing fibre with or without absorbent tissue.

Inflammation: an immune system-mediated process characterized by redness, heat, swelling, and pain at the local site.

Mammal: vertebrates with hair, three middle ear bones and mammary glands. Mammals include humans.

Skin: the outer covering of an animal body. Mammalian skin comprises three layers: (i) an epidermis layer, which is predominantly composed of keratinocytes and a small number of melanocytes and Langerhans cells (antigen presenting cells); (ii) a dermis layer, which contains nerve endings, sweat glands and oil (sebaceous) glands, hair follicles, and blood vessels and which is primarily composed of fibroblasts; and (iii) a hypodermis layer of deeper subcutaneous fat and connective tissue. The epidermis itself is made up of two layers, the outer stratum corneum and the inner epidermal basal layer, sometimes referred to as the basement membrane. The purpose of the stratum corneum is to form a barrier to protect underlying tissue from infection, dehydration, chemicals and mechanical stress.

Therapeutically-effective amount: the amount necessary to bring about a therapeutic effect.

Transdermal: passing through the dermis.

### General

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. concentration). A range of values will be understood to include all values within the range, including the values defining the range, and values adjacent to the range which lead to the same or substantially the same outcome as the values immediately adjacent to that value which defines the boundary to the range.

The following Examples are to be construed as merely illustrative and not limitative of the remainder of the disclosure in any way whatsoever. These Examples are included solely for the purposes of exemplifying the present invention. They should not be understood as a restriction on the broad summary, disclosure or description of the invention as set out above. Without further elaboration, it is believed that one skilled in the art can, using the preceding description, utilize the present invention to its fullest extent. In the foregoing and in the following examples, all temperatures are set forth uncorrected in degrees Celsius; and, unless otherwise indicated, all parts and percentages are by weight.

### EXAMPLES

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### Example 1

### Example Techniques for Ascertaining Permeability of Compositions Containing Cannabidiol.

The permeability of human skin has been studied for several decades. The skin consists of two major layers, the outer epidermis and the inner dermis. The stratum corneum (“SC”), the outermost 10-20 μm of the epidermis, is responsible for the skin's excellent diffusional resistance to the transdermal delivery of most drugs. Most of the skin's enzymatic activity lies in the basal cell layer of the viable epidermis. Fibrous collagen is the main structural component of the dermis. The skin vasculature is supported by this collagen and lies a few microns underneath the epidermis. Basically, it is here that permeation ends and systemic uptake begins. Many researchers have developed skin permeability relationships based on the physicochemical parameters (molecular weight, molecular volume, lipophilicity, hydrogen-bonding potentials, polarity, etc.) of skin penetrants. However, when dealing with transdermal administration of cannabinoids, these skin permeability relationships need to be modified to take into account the potential complications of extreme lipophilicity and concurrent metabolism of these drugs.

Selection and optimization of cannabinoids for delivery into the epidermis and dermis requires an understanding of their cutaneous metabolism. Furthermore, since skin metabolism of topical in vivo studies cannot easily be distinguished from blood, liver, or other tissue metabolism, cutaneous metabolism is better studied in vitro. However, the success of any such in vitro study depends heavily on finding ideal conditions to simulate in vivo conditions, especially in maintaining tissue viability. Thus, selection of an optimal receiver solution is critical to the success of any such in vitro studies.

A high-pressure liquid chromatography (HPLC) assay can be used for the analysis of cannabidiol in samples. An appropriate HPLC system may consist of a Waters 717 plus Autosampler, Waters 1525 Binary HPLC Pump and Waters 2487 Dual A Absorbance Detector with Waters Breeze software. A Brown-lee C-18 reversed-phase Spheri-5 μm column (220×4.6 mm) with a C-18 reversed phase 7 μm guard column (15×3.2 mm) may be used with the UV detector set at a wavelength of 215 nm. The mobile phase may comprise of acetonitrile: 25 mM phosphate buffer with 0.1% triethylamine pH 3.0 (80:20). An appropriate flow rate of the mobile phase would be 1.5 mL and 100 μL of the sample would be injected onto the column.

A PermeGear flow-through (In-Line, Riegelsville, Pa.) diffusion cell system is appropriate for the skin permeation studies. Trans-epidermal water loss can be measured (Evaporimeter EPI™, ServoMed, Sweden) after securing the skin in the cells. Pieces of skin with readings below 10 g/m2/h would be used for the diffusion studies. The skin surface in the diffusion cells would be maintained at 32° C. with a circulating water bath. An appropriate receiver solution would be HEPES-buffered Hanks' balanced salts with gentamicin (to inhibit microbial growth) containing 40% polyethylene glycol 400 (pH 7.4), and the flow rate was adjusted to 1.1 mL/h. An excess quantity of CBD would be added to the donor vehicle (propylene glycol: Hanks' buffer (80:20)) solution with and without permeation enhancers at 6% v/v, sonicated for 10 min, and then applied onto the skin. Excess quantity of the drug would be used in the donor compartment throughout the diffusion experiment in order to maintain maximum and constant chemical potential of the drug in the donor vehicle. Each cell would appropriately be charged with 0.25 mL of the respective drug solution. Samples would appropriately be collected in 6 h increments for 48 h. All the samples would appropriately be stored at 4° C. until HPLC analysis.

Drug disposition in the skin samples would be measured at the completion of the 48 h experiment. The skin tissue would be rinsed with nanopure water and blotted with a paper towel. To remove the drug formulation adhering to the surface, the skin would be tape stripped twice using book tape (Scotch®, 3M, St. Paul, Minn.). The skin in contact with the drug would be excised, minced with a scalpel and placed in a pre-weighed vial. Drug would be extracted from the skin by equilibrating with 10 mL of ACN in a shaking water bath overnight at room temperature. Samples would be analyzed by HPLC to determine CBD content in micromoles (μm) of drug per gram of wet tissue weight. Statistical analysis of the in vitro human skin permeation data could be performed using SigmaStat 2.03. A one-way ANOVA with Tukey post-hoc analysis could be used to test the statistical differences among the different treatments.

The results of such a study are expected to indicate that cannabidiol can be delivered via the topical route using compositions according to the present invention, and that siloxanes, low molecular weight alcohols, fatty alcohols and/or alkyl PEG/PPG ether increase the amounts of cannabidiol delivered into human skin.

### Example 2

### Objective:

To prepare formulations of cannabidiol with a siloxane, together with other excipients.

### Methods and Results:

First, the solubility of cannabidiol (CBD) was assessed. The powder looked granulated under the microscope. The solubility (weight to weight) of CBD was under about 3-4% in hexamethyldisiloxane (HDS) and mineral oil. The solubility in propylene glycol (PG) and ethanol was about 6-7%, although the reported solubility in ethanol is 3.5%. The solubility in oleyl alcohol (OA) was greater than 8% (did not go higher in studies) and the solubility in isopropyl alcohol (IPA) was greater than 14%. The conclusions from the solubility studies were that OA and IPA were very good solvents and it was surprising that IPA was so much better than ethanol. The solubility in HDS and mineral oil was low, so a completely nonpolar solvent does not work well to dissolve high levels of CBD, but the addition of an OH group present in a fatty alcohol really increased the CBD solubility.

Second, the CBD was dissolved at a moderate concentration in a highly volatile solvent with some nonvolatile solvents that would keep CBD in solution (non-crystalline), i.e., prevent crystallization at high concentrations (of the order of 40-50%).

Formulations:

The following formulations were prepared:
a) Form I: 5% CBD/10% OA/10% PG/10% HDS/65% IPA (some HDS was added because it has little odour, is very volatile, and reduced irritation). The residual concentration of CBD in the PG/OA would be 20%, which appeared a suitable good target. A drop of the formulation was placed on a microscope slide and there was no CBD crystallization post evaporation of highly volatile solvents. The residue remained crystal free after an hour, so more CBD was added to make a 14% CBD/9% OA/9% PG/9% HDS/59% IPA solution. The residual concentration of CBD was then 44% CBD, still no CBD crystals after evaporation. Even overnight, no crystals were observed.
b) Form II: 14% CBD/4.5% OA/13.5% PG/4.5% HDS/63.5% IPA. This solution also did not form crystals in one hour or overnight.
c) Form III: 8% CBD in just IPA. No crystals after an hour but overnight there were needle-like crystals that looked clear, not yellowish, under the microscope. The film of just liquid CBD in the microscope slide and on skin was of high friction, and probably would not be so acceptable to patients. A 10% solution in IPA applied to 1 cm² would give about a 1 Omicron thick layer (10 mg), about the thickness of stratum corneum. Made up 15% CBD in IPA and 15% CBD in 50/50 IPA/HDS with no crystals immediately.
d) Both Form I and Form II were thickened with 1% Klucel MF. Both took several minutes to become less tacky and neither of them formed crystals even after two days (samples on microscope slides). Form III was also gelled and was tacky.
e) Form IV: 3% CBD/9% PMS/88% HDS This solution was placed on a microscope slide and as the HDS evaporated the PMS was left with tiny spheres of CBD dispersed in the PMS. It was not tacky on the skin. No crystals appeared that day but overnight needle crystals appeared. Residual is 25% CBD.
f) Form V: 7.6% CBD/8% OA/8% PMS/76.4% HDS with a residual CBD of 32%. There were no crystals overnight. Added further CBD and PMS to make 10% CBD/7.7% OA/8.7% PMS/73.6HDS with a residual of 38% CBD and with similar feel and no crystals.
g) Form VI: 14% CBD/6% OA/6% PG/10% HDS/64% IPA with a residual of 54% CBD. This formulation had crystals after 48 hours. Added Klucel and only a few crystals after 48 hours. It was less tacky than the other two gels with higher OA and PG.
h) Form VII: 15% CBD/10% argan/10% HDS/65% IPA with residual of 60% CBD. A few crystals were observed after 2-3 hours. After adding Klucel, the gel had a better feel than the ones with PG and OA.
i) Form VIII: 15% CBD/5% PMS/10% OA/70% HDS. Good feel and no crystals.
j) Form IX: 10% CBD/7% argan/7% ISA/9% PMS/67% HDS. No crystals.
k) Form X: 15% CBD/13% ISA/7% PMS/66% HDS with a residual of 43% CBD. No crystals.
l) Form XI: 15% CBD/12.5% HDA/6% PMS/66.5% HDS with a residual CBD of 45%. No crystals, just droplets in PMS.
m) Form XII: 15% CBD/12.5% ODDA/6% PMS/66.5% HDS with a residual CBD of 45%. No crystals, just droplets in PMS.
n) Form XIII: 15% CBD/10% HDA/40% IPA/35% HDS with a residual CBD of 60%. No crystals. Reason for reducing IPA was to reduce potential for stinging, odour, and cooling.
o) Form XIX: 15% CBD/10% ODDA/40% IPA/35% HDS with a residual CBD of 60%. No crystals.
p) Added Klucel to Form XIII and XIX. They were not as viscous, since the HDS level was high, but they felt very good on the skin and not so tacky.
q) Form XX: 7.2% CBD/6.3% PMS/1.4% MO/1.8% IPA/83.3% HDS. No crystal of CBD and great feel with a residual CBD of 48%.
r) Form XXI: 20% CBD/10% ODDA/70% IPA with a residual CBD of 67% and no crystals.
s) Form XXII: 9.5 CBD/4.8% ODDA/57.1% EtOH/28.6% HDS with no crystals and a residual CBD of 66%.
t) Form XXIII: 10% CBD/12.5% PMS/4.5% IPA/72% HDS with good feel and no crystals with a residual CDB of 42%. Added about 4% petrolatum and had a hazy solution (from petrolatum) with no crystals.

### Example 3

### Objective:

To prepare further formulations of cannabidiol with a siloxane, together with other excipients.

### Methods:

CBD2 is an off-white powder of crystals that produced clear solutions in marked contrast to CBD1 solutions that were colored by the end of the day. None of the CBD2 solutions were colored at the end of day 1 and looked clear. The CBD2 material dissolved like the CBD1 therefore the CBD2 is CBD without the discoloration properties of CBD1.

Formulations

Formulation A (Form A)

5% CBD/2.5% HDA/1% PMS/91.5% HDS

A repeat of the acne “spray on” formulation A-7 was conducted and it behaved the same except it did not have any discoloration and was clear. It showed no signs of discoloration by the end of the day.

Tests Performed:
a) A drop on a microscope slide covered about 1 cm² and no crystals appeared until later in the day (about 4 hours later) when it was rubbed vigorously with a finger, which resulted in crystal growth.
b) Drops of Form A were placed on the skin and spread around with a finger. It dried quickly and was smooth and transparent on the skin. These results were consistent with the behaviour of A-7 with CBD1.
c) Drops of Form A were spread and rubbed lightly onto the back of the hand, and after 5 minutes a microscope slide was pressed hard against the skin and some material was transferred to the slide. Under the microscope slide there were some CBD crystals. It is a transparent film. It appears that if the film is not mechanically disturbed, crystals do not form, but with rubbing, some crystals are formed.
d) Added about 100 mg of PMS to Form A to make it about 3% PMS vs. 1%. This appeared to reduce the crystallization using the skin blot technique, but this was only a qualitative observation.

Formulation B

5% CBD/1.7% HDA/1.2% PMS/92.1% HDS

This formulation was made to determine if HDA could be reduced slightly. It appeared in all the tests to be similar to Form A.

Formulation C

5.25% CBD/1.15% PMS/1.22% IPA/92.38% HDS

The objective of this formulation was to determine whether HDA could be replaced by IPA.

Tests performed: A drop of Form C was placed on a microscope slide and it spread out to make clear film, which quickly became a white film. Under the microscope there were tiny crystals stuck together by the PMS. When placed on the skin, it turned chalky white as well. The inventors tried adding additional PMS up to about 5% but that did not end the chalkiness, although it slowed the rate down.

### Example 4

### Objective:

To determine if arlamol E (AE) or isopropyl myristate (IPM) could replace hexyldecyl alcohol (HDA), since they are both used in pharmaceutical topical products for the acne formulation 5% CBD/2.5% HDA/1% PMS/91.5% HDS.

### Summary

AE, which was initially avoided due to an intense purple colour using CBD 1, was found to be the best replacement and even superior to HAD. It did have a slight purple colour when CBD was dissolved in pure AE at the 10% level but not in the formulations using AE.

### Results:

Solubility studies: CBD dissolved at the 10% level in AE and barely 9.5% in IPM. Further exploration was not conducted due to the small amount of drug API available for non-GMP work. CBD is soluble greater than 10% but probably not in excess of 20%, as the time to dissolve additional CBD was taking considerably longer.

Five grams each of 5% CBD/2.5% AE/1% PMS/91.5% HDS and 5% CBD/1% AE/1% PMS/93% HDS formulations were made and investigated for crystal formation after evaporation of HDS. The purpose of reducing AE was to evaluate if we could further reduce AE from the 2.5% level, which did not produce crystals on a microscope slide plus or minus rubbing or no the skin as seen from an imprint on a slide pressed hard on the skin after rubbing in the formulation. After rubbing the 1% AE formulation deposited on a slide, crystal began to form rapidly. After rubbing the 2.5% AE formulation one could observe many very tiny (smaller than a period at 100×) droplets (no crystals). Since the formulation minus CBD did not produce these droplets, it was hypothesized that the droplets are “supersaturated CBD in AE”. Without rubbing the droplets are not created and the formulation looks like a clear film.

Five grams of a 5% CBD/2.5% IPM/1% PMS/1% IPA/90.5% HDS formulation were made. IPA was added to completely dissolve the CBD. This formulation produced crystal growth rapidly as the formulation was rubbed while drying on a slide as contrasted with the AE formulation.

Five grams of a 10% CBD/4% AE/1% PMS/1% IPA/84% HDS formulation were made (IPA added to completely dissolve the CBD). This formulation did not produce crystals of CBD upon evaporation and rubbing on a slide although it had a reduced ration of CBD:AE. The residual solution of CBD in AE would be 71%.

Formulation Recommendations are:

5% CBD/2% AE/1% PMS/92% HDS

10% CBD/4% AE/1% PMS/1% IPA/84% HDS

### Example 5

### Objective:

To test several more formulations at the 5%, 10%, and 15% CBD concentrations.

Methods:

The acne formulations were alcohol (isopropyl alcohol [IPA]) based to allow for thickening with Klucel and silioxane (hexylmethyldisiloxane [HDS]) based for spray on formulations. The psoriasis formulations were siloxane based and thickened with polymethylsiloxane 10⁶ cSt (PMS). All the formulations would be suitable for human studies, and under microscope evaluation post evaporation all formulations did not crystalize CBD. The residual solubilizer was 2-hexyldecyl alcohol (HDA) and residual concentrations were 60% to 67%.

### Formulations

### Acne “Gels”

A-1: 5% CBD/2.5% HDA/50% IPA/41% HDS/1% KlucelMF

At 1% Klucel this gel and all the 1% Gels were basically thickened such that they could be applied from a dropper container to spread on the skin. Additional Klucel was added to this formulation, which became much stiffer.

A-2: 5% CBD/3.33% HDA/50% IPA/40.67% HDS/1% KlucelMF

A-3: 5% CBD/3.33% HDA/75% IPA/15.67% HDS/1% KlucelMF

A-4: 10% CBD/6.67% HDA/75% IPA/7.33% HDS/1% KlucelMF

A-5: 15% CBD/10% HDA/70% IPA/4% HDS/1% KlucelMF

A-6: 15% CBD/7.5% HDA/70% IPA/6% HDS/1.5% KlucelMF

This formulation had 0.5% more Klucel and was more viscous.

Acne “Spray On”

A-7: 5% CBD/2.5% HDA/1% PMS/91.5% HDS

A-8: 10% CBD/5% HDA/1% PMS/84% HDS

A-9: 15% CBD/7.5% HDA/1% PMS/1% IPA/1% D5/74.5% HDS

1% IPA was added because the CBD was not quite soluble at 15% without IPA.

Observations of the formulations indicated that the formulations (which were not light protected) with alcohol tended to be darker with time than those without alcohol.

Psoriasis Formulations (similar to the acne spray but with more PMS)

P-1: 5% CBD/2.5% HDA/5% PMS/87.5% HDS

P-2: 10% CBD/6.67% HDA/5% PMS/78.33% HDS

P-3: 15% CBD/7.5% HDA/5% PMS/1% IPA/71.5% HDS

P-4: 15% CBD/7.5% HDA/10% PMS/1% IPA/66.5% HDS

As for the acne spray on formulations 1% IPA was employed for the 15% CBD formulations.

### Example 6

A Randomised, Double-Blind, Vehicle-Controlled Study of the Safety and Tolerability of BTX 1204 in Patients with Mild to Moderate Atopic Dermatitis. This study will be carried out to determine the safety and tolerability of BTX 1204 in participants with mild to moderate atopic dermatitis (AD). This is will be a multi-centre, double-blind, vehicle-controlled, parallel-group study.

### Methodology:

Test Product, Dose and Mode of Administration, Batch Number:
Test Product: BTX 1204-4% (w/w) Solution. Contains the active pharmaceutical ingredient, cannabidiol (CBD; 2-[(1R,6R)-6-isopropenyl-3-methylcyclohex-2-en-1-yl]-5-pentylbenzene-1,3-diol).
Administration: One or 3 mL of the study drug was applied topically to the face once (QD) or twice (BID) daily (at about the same time each day) using an applicator swab.
Batch Number: PPP.17.566
Single-dose on Day 1, then multiple dosing starting on Day 8 for 14 days to Day 21

TABLE 2
Composition of 4% BTX 1204

| Ingredients | 4% Solution (% w/w) |
|---|---|
| Hexamethyldisiloxane (HDS) | 94.05 |
| Polypropylene Glycol-15 (PPG-15) Stearyl Ether | 1.95 |
| Cannabidiol (CBD) | 4.0 |

This dose level is well below that tested and shown to be well-tolerated in a 28-day study previously carried out by the present laboratory in minipigs. Specifically, the NOAEL for dermal tolerability of BTX 1503 5% (w/w) on the skin of minipigs was 3.0 mg/cm²/day (150 mg/kg/day), which is ˜9 times the daily dose proposed in the present study. In addition, based on the ratio of the mean Cmax observed in the 28-day minipig study to the mean Cmax observed in a Phase 1a study for acne treatment using BTX 1503 5% (w/w), there was >300 times the level of CBD in the minipigs than the Phase 1a acne study, with no observed effect in either study.

Each milliliter of the BTX 1204 4% (w/w) Solution contains 30.0 mg of CBD. Participants will apply 3 mL of the BTX 1204 4% (w/w) Solution twice daily resulting in a maximum of 180 mg of CBD applied daily.

Number of Participants: 24 participants to BTX 1204 4% (w/w) Solution and 12 participants to Vehicle Solution. This study included male and female participants who were between 18 and 65 years of age (inclusive). Participants were in good general health without clinically significant disease.

Safety will be the primary outcome measure. The safety outcome measures to be assessed are:
Adverse events (AEs) will be monitored from time of consent through the end of study.
Complete blood count (CBC), chemistry, and urinalysis conducted at Baseline and at Day 29.
Urine drug tests for tetrahydrocannabinol (THC) levels conducted at the Baseline (Day 1), Day 8, Day 15, and Day 29 visits to evaluate for levels of THC.
Signs of AD on the target lesion (erythema, exudation, excoriation, induration/papulation, and lichenification) obtained at the Baseline (Day 1), Day 8, Day 15, and Day 29 visits.
Cutaneous tolerability (erythema, scaling, dryness, burning/stinging, and irritant/allergic contact dermatitis) will be collected at Baseline, Day 8, Day 15, and Day 29 and graded using the following scale: 0, None; 1, Slight; 2, Moderate; 3, Severe.
Participant reports of burning/stinging obtained daily on a Patient Diary.
Participant's reports of pruritus obtained daily on a Patient Diary.
Blood samples taken pre-dose (15 minutes before dosing) on Day 1 (Baseline) and on the morning of Day 29 to assess the plasma levels of study drug.

Assessments of the pharmacologic effect of BTX 1204 will be evaluated using the Investigator's Static Global Assessment (ISGA) on the target lesion obtained by the treating dermatologist(s) at the Day 1 and Day 29 Visits and a change from Baseline in the target lesion size at Day 29.

### Method

Approximately 36 participants randomised 2:1 (24 participants to BTX 1204 4% (w/w) Solution and 12 participants to Vehicle Solution) with AD will be enrolled. The selected sample size is based on having appropriate sensitivity to observe a safety signal. Thirty-six (36) participants, 24 receiving active BTX 1204 4% (w/w) Solution and 12 receiving Vehicle Solution, will be adequate to detect if there are any systemic safety or tolerability concerns.

Participants will begin screening to determine eligibility to participate in the study. Informed consent, medical history/review of systems, demographics, height and weight will be obtained. A target lesion will be identified based on the inclusion criteria. Measurement of the target lesion and total body surface area (BSA) of AD involvement will be obtained. A urine drug screen (UDS) will occur. Signs of AD and ISGA for the target lesion will be assessed at Screening for eligibility.

A target lesion will be selected, based on the eligibility criteria, and measured. The length at the highest to lowest point and the width across the widest part will be measured in centimeters.

The total body surface area (BSA) of atopic dermatitis involvement will be obtained. The BSA can be approximated using the Rule of 9 s or the palm (1%) method.

Signs of AD on the target lesion will be obtained (see Table 3).

An ISGA on the target lesion will be conducted. The participant must have an ISGA score of mild (2) or moderate (3) (see Table 4). The ISGA assesses the overall status of the target lesion at the time of the assessment. The ISGA is to be conducted by the same investigator/sub-investigator at both visits. No comparisons are made to previous assessments.

TABLE 2
Signs of Atopic Dermatitis (Paller)

| | | |
|---|---|---|
| Score | Grade | Definition |

Erythema (redness)

| | | |
|---|---|---|
| 0 | None | No redness |
| 1 | Mild | Mildly detectable erythema; pink |
| 2 | Moderate | Dull red; clearly distinguishable |
| 3 | Severe | Deep, dark red; marked and extensive |

Exudation (oozing and crusting)

| | | |
|---|---|---|
| 0 | None | No oozing or crusting |
| 1 | Mild | Minor or faint signs of oozing |
| 2 | Moderate | Definite oozing or crusting |
| 3 | Severe | Marked and extensive oozing or crusting |

Excoriation (evidence of scratching)

| | | |
|---|---|---|
| 0 | None | No evidence of excoriation |
| 1 | Mild | Mild excoriation |
| 2 | Moderate | Definite excoriation |
| 3 | Severe | Marked, deep, or extensive excoriation |

Induration/papulation

| | | |
|---|---|---|
| 0 | None | None |
| 1 | Mild | Slightly perceptible elevation |
| 2 | Moderate | Clearly perceptible elevation but not extensive |
| 3 | Severe | Marked and extensive elevation |

Lichenification (epidermal thickening)

| | | |
|---|---|---|
| 0 | None | No epidermal thickening |
| 1 | Mild | Minor epidermal thickening |
| 2 | Moderate | Moderate epidermal thickening; accentuated skin lines |
| 3 | Severe | Severe epidermal thickening; deeply accentuated skin |
| | | lines |

TABLE 4
Investigator's Static Global Assessment
(ISGA) on the Target Lesion

| Score | Severity | Definition |
|---|---|---|
| 0 | Clear | Minor residual discoloration; no erythema or |
| | | induration/papulation, no oozing/crusting |
| 1 | Almost Clear | Trace faint pink erythema, with barely perceptible |
| | | induration/papulation, no oozing/crusting |
| 2 | Mild | Faint pink erythema with mild induration/ |
| | | papulation, no oozing/crusting |
| 3 | Moderate | Pink-red erythema with moderate induration/ |
| | | papulation with or without oozing/crusting |
| 4 | Severe | Deep/bright red erythema with severe induration/ |
| | | papulation, with oozing/crusting |

Within 14 days after the Screening Visit, Baseline assessments for safety (CBC, chemistry, and urinalysis) will be obtained on Day 1. A blood sample will be obtained for study drug blood levels within 15 minutes prior to study drug application. If the participant is eligible to participate, Screening and Baseline may occur at the same visit. If the Screening Visit and Baseline Visit is not concurrent, UDS, Signs of AD, and ISGA will be repeated at the Baseline Visit.

For all participants, a CBC, chemistry, and urinalysis will be conducted at the Baseline and Day 29 visits. If an abnormal laboratory assessment is returned from the Baseline assessments, consideration will be given by the investigator as to the continued participation of the participant in the study.

Blood samples will be taken per standard venipuncture techniques and sent to the local lab for analysis. Samples for CBC, chemistry and urinalysis will be collected at approximately the same time in the morning during the required visits. The following will be assessed:

CBC: White blood cell (WBC) count (with automated differential for absolute neutrophils, lymphocytes, monocytes, eosinophils, and basophils), red blood cell (RBC) count, haemoglobin, hematocrit, mean corpuscular volume (MCV), mean corpuscular haemoglobin (MCH), mean corpuscular haemoglobin concentration (MCHC), and platelet count

Chemistry: Glucose, albumin, total protein, calcium, sodium, potassium, chloride, CO2 (bicarbonate), blood urea nitrogen (BUN), creatinine, alkaline phosphatase, alanine amino transferase (ALT), aspartate amine transferase (AST), and total bilirubin

Urinalysis: Color, clarity, specific gravity, pH, protein, glucose, leukocyte and esterase. If results are abnormal, the sample will be further assessed using microscopic analysis for red blood cells, white blood cells, squamous epithelial cells, and culture.

All participants will have a blood sample taken within 15 minutes before dosing on the Day 1 Visit and another at the Day 29 Visit to measure plasma levels of CBD. Plasma samples will be analysed using a validated liquid chromatography-tandem mass spectrometry (LC-MS/MS) method. The limit of detection is 0.2 ng/mL.

Participants will be randomised 2:1 using the Interactive Voice Response System (IVRS)/Interactive Web-based Response System (IWRS) to receive either active BTX 1204 4% (w/w) Solution or Vehicle Solution. Participants will receive their first dose of study drug applied by the site staff and will be observed in the clinic for one hour after application. Cutaneous tolerability assessments will be conducted at one hour after the first application. Participants will be given one week of study drug and instructed in the proper application to cover their target AD lesion and surrounding skin. Participants will be provided with a diary to record their daily study drug application and daily recording of burning/stinging and pruritus.

Participants will return to the clinic on the morning of Day 8 for cutaneous tolerability assessments and a UDS for presence of THC prior to the application of study drug. Participants will also be queried for AEs and changes in concomitant medications. Diaries and study drug will be returned and reviewed for compliance and daily assessment of burning/stinging and pruritus. The site will obtain Signs of AD. The participant will then apply their morning dose of study drug during the visit for the clinical site to confirm correct application techniques. Another week of study drug will be dispensed.

Participants will return to the clinic on the morning of Day 15 for cutaneous tolerability assessments and a UDS for presence of THC. Participants will also be queried for AEs and changes in concomitant medications. Diaries and study drug will be returned and reviewed for compliance and daily assessment of burning/stinging and pruritus. The site will obtain Signs of AD. The participant will then apply their morning dose of study drug during the visit for the clinical site to confirm correct application techniques. Two weeks of study drug will be dispensed along with the diary for the next 14 days of the study. The final study drug application will be the p.m. application on Day 28.

Participants will return to the clinic on the morning of Day 29 for safety assessments; blood samples for CBC and chemistry, urine samples for urinalysis, cutaneous tolerability assessments, and UDS for the presence of THC. Participants will be queried for AEs and changes in concomitant medications. Diaries and study drug will be returned and reviewed for compliance and daily assessment of burning/stinging and pruritus. The site will obtain Signs of AD. The study investigator will conduct an ISGA. A blood sample will be obtained for study drug blood levels.

The following medications, treatments, and procedures are prohibited for all participants during the study.
Use of any topical agent other than study drug, including moisturizers, creams, topical antibiotics or sunscreens on the target lesion. Once the subject is enrolled, non-target lesions may be treated with topical corticosteroids but not topical antibiotics. NOTE: If a subject gets an infection during the study on the target or non-target lesions that require topical antibiotics, the subject can be treated and allowed to remain on the study. The antibiotic use will be noted as a deviation.
Use of any oral medication for the treatment of AD, including oral antibiotics. If a subject gets an infection during the study that requires oral antibiotics, the subject can be treated and allowed to remain on the study. The oral antibiotic use will be noted as a deviation.
Use of systemic corticosteroids (inhaled corticosteroid≤1000 μg daily dose is acceptable) or anti-inflammatory drugs (NSAIDs are permitted).
Photodynamic therapy.

Participants should limit exposure of the treatment area to sunlight during the study. Participants must not shower or wash the study application area for 4 hours after application of study drug. Participants should avoid swimming and heavy exercise for 4 hours after application of study drug.

### Statistical Methods

All statistical processing will be performed using SAS® unless otherwise stated.

### Safety Analyses

All participants who receive at least one confirmed dose of study drug, and have at least one post-Baseline assessment will be included in the safety analyses.

The mean, standard deviation, median and range will be calculated for the change from Baseline in each Sign of AD (erythema, exudation, excoriation, induration/papulation, and lichenification) at each timepoint (Day 8, Day 15, and Day 29). In addition, a total score will be calculated based on the sum of each of the Signs of AD times the score (0, 1, 2, or 3; max score of 15) and the change from Baseline will be summarised for each timepoint.

Cutaneous tolerability scores for each parameter (erythema, scaling, dryness, burning/stinging, and irritant/allergic contact dermatitis) will be summarised for each visit. In addition, the change from Baseline in the mean scores will be summarised for each visit.

Summaries of the amount of burning/stinging and pruritus reported by the participants in the daily Patient Diary will be summarised using daily means by treatment. Graphic presentations will be prepared to display the changes in burning/stinging and pruritus over time.

Changes in laboratory parameters from Baseline to Day 29 will be summarised using shift tables to evaluate for trends. Abnormal laboratory findings will be summarised and listed by participant.

Concomitant medications will be mapped to ATC Level 2 using the WHODrug dictionary. The number and percentage of participants reporting each medication will be summarised. Medications taken by each participant will be listed.

Drug Levels: Blood levels of study drug will be summarised for Baseline and Day 29. The mean, standard deviation (SD), median and range will be presented.

Demographics: Demographics/Baseline characteristics will be summarised by age, gender, race, ethnicity, height, weight, target lesion size, and BSA of AD.

ISGA: The change from Baseline for ISGA on the target lesion will be assessed on Day 29. The mean, SD, median, and range will be presented. The proportion of participants with an ISGA target lesion score of clear (0) or almost clear (1) and a decrease of 2-grades or more will be presented.

Target Lesion Size: The change from Baseline in the size of the target lesion will be determined.

### Example 7

Study of skin permeation and delivery measurements from cannabidiol formulations. The primary objective of the study was to determine the rate and extent of in vitro skin permeation of cannabidiol (the “Active”) into and through cadaver skin using a Franz diffusion cell system. Flux was measured over a period of 48 hours after application of the formulations.

TABLE 5
Formulations
Formulation

A: 2.5 wt % cannabidiol
B: 5.0 wt % cannabidiol
C: 2.5 wt % cannabidiol
D: 5.0 wt % cannabidiol

Intact human cadaver skin was purchased from the New York Firefighter's Skin Bank (“NYFFSB”, NY, NY). The skin tissue was dermatomed by the tissue bank to a thickness of some 250 μm and shipped frozen on dry ice. Upon receipt of the donor skin, the skin pieces were stored at −20° C. until used. Prior to use, the skin pieces were removed from the freezer and allowed to thaw fully at ambient temperature.

The following equipment was used during the course of the study:
Diffusion Cells. 24 diffusion cells with 3.3 ml receptor volume and a 0.55 cm² receptor fluid exposure surface area.
Stirring Dry Block Heaters. Reacti-Therm #18823 stirring dry block heaters were used to maintain the receptor fluid at 32±0.5° C. with constant stirring throughout the study.
The analysis was carried out with an Agilent 1260 HPLC unit with a G16120 MS detector, ID #: TM-EQ-069.
Tritiated water signals were analyzed with a PerkinElmer MicroBeta TriLux 1450 Liquid scintillation counter (“LSC”). ID #: TM-EQ-047.

The following materials and reagents were used for the study.

TABLE 6
Materials and reagents used in the study

| Material | Supplier: | Catalog#: | Lot#: |
|---|---|---|---|
| Cannabidiol | Botanix | | 9100042 |
| Methanol | FisherSci Optima | A456-4 | 173438 |
| Water | Millipore | WX0008-1 | 56160 |
| Hydroxypropyl-β- | TCI | H0979 | PJT4B |
| cyclodextrin (HPBCD) | | | |
| Brij O20 | Croda | 436240 | MKBP0994V |
| Formic Acid | Sigma Aldrich | 56302 | BCBQ3264V |
| Ammonium formate | Sigma Aldrich | 17843 | BCBP1919V |
| ³H Water | Perkin Elmer | Net001B001 | 1738956 |
| PBS (10X diluted to | Quality | 119-069-131 | 721676 |
| 1X) | Biologicals | | |
| Zorbax Eclipse PAH | Agilent | | |
| narrow bore C18 RR | | | |
| (2.1 × 100 mm, | | | |
| 3.5 um, 600 bar) | | | |

A liquid chromatography mass spectrometry (“LC/MS”) analytical method was used to detect for cannabidiol (“CBD”).

### Preparation of Mobile Phases

Mobile Phase A: Mobile Phase A was prepared by first transferring 1.0 ml of formic acid (Sigma Aldrich: 56302) into a 2 L media bottle. 1 L of HPLC grade water (Millipore: WX0008-1) was then measured in a volumetric cylinder and the contents transferred into the 2 L media bottle. Finally, 630.6 mg of ammonium formate was then weighed and also transferred to the media bottle. The mixture in the media bottle was then shaken until the contents were fully dissolved. Mobile Phase A was stored for less than one week during the course of the analysis.

Mobile Phase B: Mobile Phase B was prepared by transferring 1.0 ml of Formic acid (Sigma Aldrich: 56302) into a 2 L media bottle. 1 L of HPLC grade methanol (Millipore: AX-0145P) was then measured in a volumetric cylinder and the contents transferred into the 2 L media bottle. Finally, 630.6 mg of ammonium formate was then weighed and also transferred to the media bottle. The mixture in the media bottle was shaken until the contents were fully mixed. Mobile Phase B was stored for less than one week during the course of the analysis.

### Preparation of Stock Solution and Calibration Standards

Individual calibration standards were prepared for CBD. A CBD “Stock Solution” was prepared by first weighing 4 mg of CBD with an analytical balance in a glass vial. The vial was then tared on the balance and 4 ml of dimethyl sulfoxide (“DMSO”) was introduced in to the glass vial with a pipettor. The vial was reweighed. The vial was then removed from the analytical balance and capped. The capped vial was vortexed and sonicated using an ultrasonication bath until the CBD was fully dissolved.

The above procedure was used to make a 1 mg/ml Stock Solution for CBD. Further calibration standards were prepared through serial dilution. In each serial dilution, 300□l of the preceding calibration standard was diluted with 1200 μl of DMSO. Eight calibration standards were prepared. The CBD concentration in each of the calibration standards is shown in Table 7 below.

TABLE 7
Calibration standards and the corresponding
concentration of the CBD.

| Active | CBD | |
|---|---|---|
| Calibration standard | Conc (μg/ml) | |
| Stock Solution | 1000 μg/ml Stock Solution | |
| Cal 2 | 200 | μg/ml |
| Cal 3 | 40 | μg/ml |
| Cal 4 | 8 | μg/ml |
| Cal 5 | 1.6 | μg/ml |
| Cal 6 | 0.32 | μg/ml |
| Cal 7 | 0.064 | μg/ml |
| Cal 8 | 0.0128 | μg/ml |

The CBD was first prepared in a Stock Solution. Separate calibration standards were then prepared for by serial five-fold dilutions with DMSO. Standards Cal3-Cal8 were used for the calibration curves.

### Preparation of Sample Solution

The study samples were collected during the permeation studies. No further preparation was done on the samples prior to analysis.

### Chromatographic Parameters

An outline of the method details is provided in Table 8 below.

TABLE 8
Chromatographic parameters for CBD detection.

| Instrument: | 1200-HPLC/UV/MSMS Xevo TQD | |
|---|---|---|
| Column: | Zorbax Eclipse PAH narrow bore C18 RR (2.1 × | |
| | 100 mm, 3.5 um, 600 bar) | |
| | Guard column: PAH 12.5 × 3.5 μm | |
| Mobile phase: | A: Water with 0.1% FA, 10 mM NH₄HCO₂ | |
| | B: Methanol with 0.1% FA, 10 mM NH₄HCO₂ | |
| Gradient: | Time (minutes) | % B |
| | 0 | 70% |
| | 1.0 | 70% |
| | 5.0 | 95% |
| | 7.0 | 95% |
| | Post time | 3 min |
| Flow rate: | 1.0 ml/min | |
| Column | 50° C. | |
| temperature: | | |
| MS detection: | ESI(+)-MRM: m/z 315.2 > 193.1 | |
| | Collision energy: 20 V | |
| Injection volume: | 5 μl | |
| Diluent for | DMSO | |
| standards: | | |
| Retention time: | CBD | ~4.2 minutes |

### Calculation

After the LC/MS testing was complete, the samples were analyzed using Chemstation software. The AUCs of the CBD peaks were recorded and converted to □g/ml values using a calibration curve developed from the calibration standards' AUC values and known concentration values. These μg/ml values were imported into the study results Excel workbook. These concentrations were then multiplied by the receptor volume (3.3 mL) and divided by the surface area of the skin exposed to the receptor fluid (0.55 cm²) for an end cumulative amount in μg/cm². For receptor fluid time points greater than 4 hrs, this μg/cm² value was corrected for the sample aliquot volumes which were removed to compensate for the dilution caused by replacing the sample volume with fresh buffer solution. As an example, for the second time point at 10 hrs, the dilution factor (300 μl aliquot/3.3 ml receptor volume or 1/11) is multiplied by the μg/cm² value calculated for the 4 hr time point, the result of which is then added to the μg/cm² concentration which is calculated using the 10 hr AUC value. Equation 1 outlines the correction value for the dilution effect.

$$Cumulative amount ( in μ g {cm}^{- 2} ) = \frac{\begin{matrix}( AUC + \\ Σ ( AUCs of previous \\ timepoints ) \times \\ \frac{sample volume}{receptor volume} ) \times receptor vol\end{matrix}}{( calibration slope \times surface area )} \qquad Equation \#1 A ( Dilution correction )$$

### Receptor Fluid

The receptor fluid (the “Receptor Fluid”) consisted of phosphate buffered saline (“PBS”), sourced from Quality Biologicals with 0.01 wt % NaN₃ (added as a preservative), 4 wt % hydroxypropyl-β-cyclodextrin (added to increase solubility of the Actives) and 1 wt % Brij 020. The PBS was supplied as 10× concentration and was diluted to 1× concentration prior to the study by volumetrically adding distilled water at a 9:1 water to concentrated PBS ratio. The solubility of CBD in the Receptor Fluid was previously measured to be ˜>50 μg/ml and was determined to be sufficient to maintain sink conditions throughout the study.

After mixing the Receptor Fluid, degassing of the Receptor Fluid was accomplished according to Tioga's Standard Operating Procedure (“SOP”) SOP Lab.007.1 ‘Degassing of receptor fluid for diffusion studies’. Receptor Fluid was filtered through a ZapCap CR 0.2 μm membrane under vacuum; the Receptor Fluid, so filtered, was stirred for an additional 20 minutes under vacuum.

### Skin Preparation

Human cadaver skin from NYFFSB was prepared as follows prior to assembling the diffusion cells.
The cadaver skin piece was removed from the freezer and allowed to defrost in a Bio-safety hood for 30 minutes. Prior to opening the package, a visual inspection was used to confirm that the skin piece had been thoroughly defrosted.
The cadaver skin piece was removed from the package and placed in a distilled water bath for 30 seconds to wash off any cryoprotectants from the skin. The skin was then removed from the water bath and placed in a Bio-safety hood. The exterior surface of the skin was patted dry with a KimWipe, sprayed with fresh PBS, and then patted dry again.

### Assembling the Franz-type Diffusion Cells

Glass FDCs with a 3.3 ml receiver volume and 0.55 cm² diffusional area, custom fabricated to Tioga specifications, were used. Once the skin had been defrosted and washed, the FDCs were prepared as follows:
The receptor wells were filled with degassed Receptor Fluid using a pipette.
A 6 mm by 3 mm diameter Teflon coated magnetic stir bar was introduced into each receptor well.
The defrosted and washed cadaver skin pieces were examined and only areas of even thickness and with no visible surface damage were used.
The skin piece was cut into approximately 2 cm×2 cm squares using skin scissors. The square sizes were adjusted as necessary according to the shape and dimensions of the skin piece, but were selected to be approximately uniform in size among all FDCs.
A skin piece was centered on each inverted donor compartment, with the stratum corneum (“SC”) side contacting the donor compartment.
The donor and receptor well compartments were then aligned and clamped together with a pinch clamp, ensuring that the skin pieces were centered between both donor and receptor wells.
Additional Receptor Fluid was added as necessary. Air bubbles in the receptor well, if any, were removed by tilting the FDC assembly such that the air escapes along the sample port. Receptor wells were filled with approximately 3.3 ml of Receptor Fluid.
The assembled FDCs were placed into stirring dry block heaters which were preheated to 32° C. The Receptor Fluid was continuously agitated via the magnetic stir bar.
After 20 minutes, the surface of the skin in each FDC was examined. If the skin appeared wet or showed signs of sweating, the cell was discarded.
Approximately 24 FDCs were assembled from the skin piece.

### Membrane Integrity Check

Once the FDCs were assembled, the barrier integrity of the skin pieces was tested prior to application of the test articles by a measurement of the transdermal flux of tritiated water according to Tioga SOP Lab.011.1, as outlined following:
Into 10 ml of deionized (“DI”) water was introduced 25 μl of 1 mCi/ml water (the resulting sample was termed “Tritiated Water”).
An aliquot of 150 μl of Tritiated Water was introduced into each FDC donor well.
After 10 minutes, the Tritiated Water was removed from each FDC donor well using a pipette and the skin surface tapped dry using a KimWipe.
The receptor well of each FDC was agitated for an additional 1 hour after the Tritiated Water had been removed from each donor well.
After the 1 hour of agitation, a 300 μl aliquot was abstracted from each FDC receptor well and placed into a well in a microtiter plate.
600 μL of scintillation cocktail (Ultima Gold from Perkin Elmer) was then added to each sample aliquot in the microtiter plate.
The tritium (³H) content of each sample aliquot was measured using a liquid scintillation counter (“LSC”—PerkinElmer MicroBeta TriLux 1450).
After LSC analysis was complete, results were analyzed. Any FDCs showing anomalously high water flux were discarded.
The remaining FDCs were ranked according to the magnitudes of the measured tritiated water flux values. Test articles were then assigned to the batch of FDCs such that the replicates for each test article are each applied to a skin piece with nearly equivalent average tritiated water flux values. The ranking of skin pieces was carried out separately for each substrate.
The entire volume of Receptor Fluid was removed from each FDC and replaced with fresh Receptor Fluid.
The FDCs were finally placed into preheated dry block heaters.

### Test Article Application Procedure

After the membrane integrity tests were complete and the cells appropriately sorted, samples of the test articles were then applied to the stratum corneum of the skin. A one-time dosing regimen was used for this study. Donor cells were left uncapped during the experiment. The dose of the Active applied per cell and corresponding formulation is shown in Table 9 below.

TABLE 9
CBD dose per cell for the applied test articles.

| Study | | Dose of formulation | |
|---|---|---|---|
| Arm | Formulation | applied per cell | CBD dose |
| 1 | A: 2.5 wt % cannabidiol | 5 μl | 173.9 μg/cm² |
| 2 | B: 5.0 wt % cannabidiol | 5 μl | 340.9 μg/cm² |
| 3 | C: 2.5 wt % cannabidiol | 5 μl | 173.9 μg/cm² |
| 4 | D: 5.0 wt % cannabidiol | 5 μl | 340.9 μg/cm² |

The dose assumes a specific gravity of 0.75 for the formulations, and also assumes 100% of the applied 5 μl of the formulation remains on the skin after spreading the formulation across the skin surface using a glass rod.

“Blank” undosed FDC cells were also set up to test for background signal noise. The background noise measured from these “blank” cells had negligible AUC for CBD.

### Sampling of Receptor Fluid

Using a graduated Hamilton type injector syringe, a 300 μl aliquot was abstracted from the sampling port of each FDC at each of 4, 10, 24 and 48 hours. Fresh Receptor Fluid was added to each receptor well to replace the volume of fluid abstracted. Each abstracted aliquot was introduced into a well in a 96-well microtiter plate.

Samples were stored in a refrigerator at 4-8° C. prior to LC/MS analysis. Samples were analyzed within 5 days of collection.

### Skin Extraction

At 48 hours, a 200 ul aliquot of 50 vol %/50 vol % water/ethanol was dispensed in the donor compartment of each FDC. This “washing solution” was allowed to sit for 5 minutes, after which it was removed. The skin was then tapped dry and tape stripped three times with cellophane tape, each tapestripping consisting of applying a piece of cellophane tape to the skin with light pressure and peeling off the tape, thereby systematically removing the upper most layers of the stratum corneum. The tape strips were discarded.

After tape stripping was complete, the remaining skin was split into epidermal and dermal compartments by using a pair of spatulas. If necessary, the skin was placed on a hot plate set at 60° C. for one minute to help facilitate the separation of the skin. The epidermal and dermal compartments were then separately placed into glass vials, into which 3 ml of DMSO was added to extract the CBD from the tissue. The skin pieces were then incubated at 40° C. for 24 hours with gentle agitation. After the 24 hour incubation period, samples were collected from the extraction solvent and analyzed via LC/MS detection.

### Analyses of Samples

The samples abstracted from the receptor wells were then analyzed using the MS method outlined above. The concentrations of CBD were assayed and reported in each case.

### Results

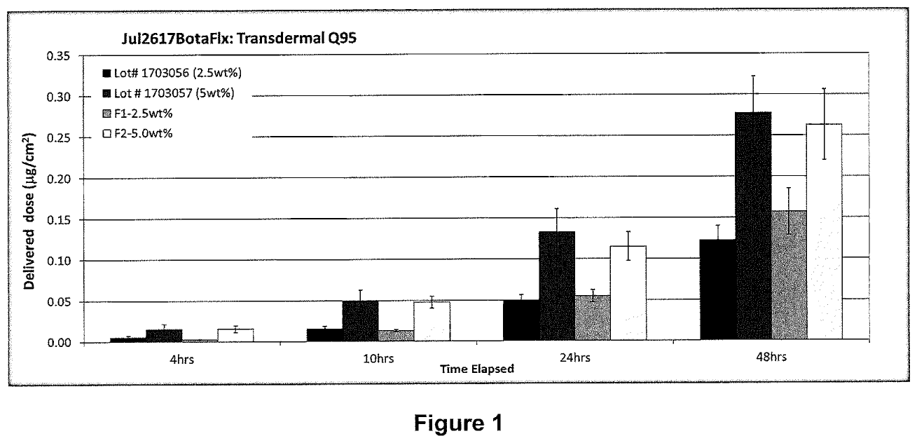

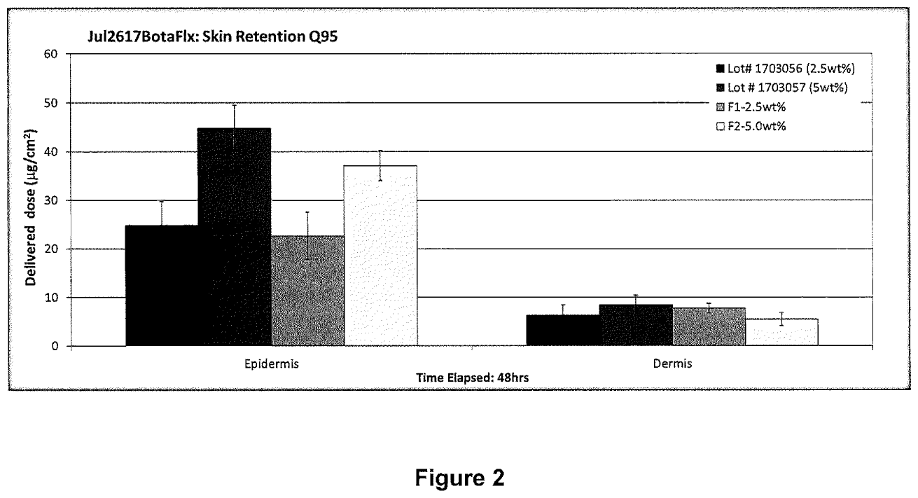

The accumulated dose of CBD at each of the time points is shown in FIGS. 1 and 2.

TABLE 10
Total accumulated dose (in μg/cm²) of CBD delivered over time.
Delivered dose (μg/cm²)

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| 4 | 0.005 | 0.015 | 0.022 | 0.016 |
| 10 | 0.015 | 0.049 | 0.013 | 0.048 |
| 24 | 0.049 | 0.133 | 0.055 | 0.115 |
| 48 | 0.122 | 0.278 | 0.157 | 0.263 |
| Epidermis | 24.921 | 44.884 | 22.641 | 37.156 |
| Dermis | 6.283 | 8.408 | 7.739 | 5.474 |
| Time (h) | Std Err | Std Err | Std Err | Std Err |
| 4 | 0.002 | 0.006 | 0.000 | 0.004 |
| 10 | 0.004 | 0.013 | 0.002 | 0.007 |
| 24 | 0.007 | 0.028 | 0.007 | 0.018 |
| 48 | 0.018 | 0.044 | 0.028 | 0.043 |
| Epidermis | 4.777 | 4.648 | 4.886 | 3.111 |
| Dermis | 2.187 | 2.023 | 1.055 | 1.395 |

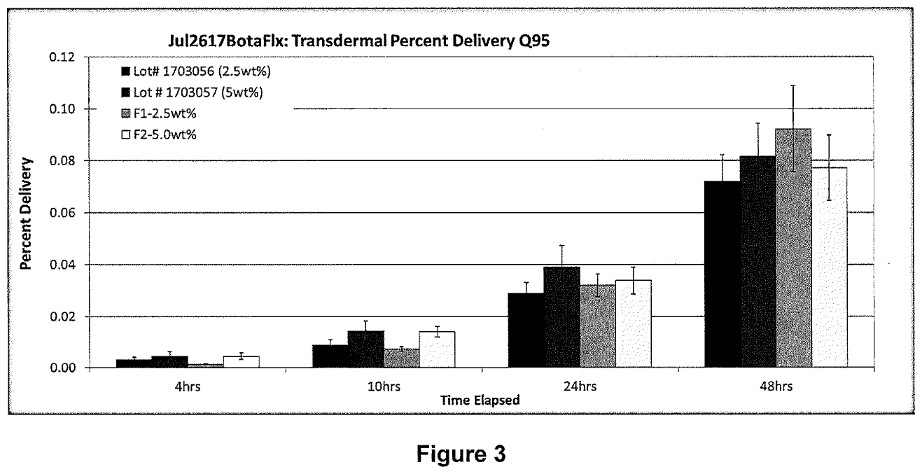

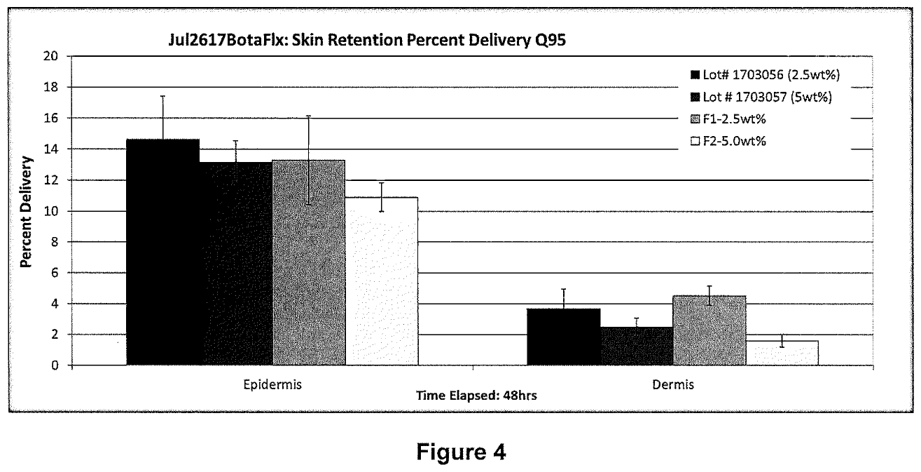

The percent delivery of CBD (taking into account the 5 μl applied dose and the formulated concentration of CBD in the formulation) at each of the time points is shown in FIGS. 3 and 4.

TABLE 11
Percent delivery of CBD delivered over time.
Percentage Delivery

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| 4 | 0.003 | 0.004 | 0.001 | 0.005 |
| 10 | 0.009 | 0.014 | 0.007 | 0.014 |
| 24 | 0.029 | 0.039 | 0.032 | 0.034 |
| 48 | 0.72 | 0.081 | 0.092 | 0.077 |
| Epidermis | 14.620 | 13.166 | 13.283 | 10.899 |
| Dermis | 3.686 | 4.540 | 7.739 | 1.606 |
| Time (h) | Std Err | Std Err | Std Err | Std Err |
| 4 | 0.001 | 0.002 | 0.000 | 0.001 |
| 10 | 0.002 | 0.004 | 0.001 | 0.002 |
| 24 | 0.004 | 0.008 | 0.004 | 0.005 |
| 48 | 0.010 | 0.013 | 0.017 | 0.013 |
| Epidermis | 2.802 | 1.363 | 2.866 | 0.913 |
| Dermis | 1.283 | 0.593 | 0.619 | 0.409 |

The percent delivery assumes a specific gravity of 0.75 and that 100% of the 5 μL applied dose remains on the skin after spreading the formulation with the glass rod. Percent delivery takes into account the varying concentrations of CBD present in each formulation.

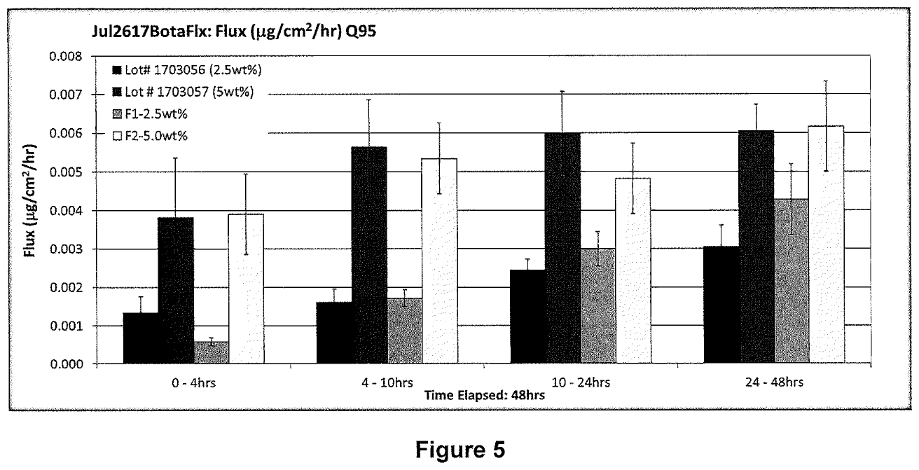

The flux of CBD between each of the time points is shown in FIG. 5.

TABLE 12
Flux of CBD over time (in μg/cm2/hr).
Flux (μg/cm²/h)

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| 0-4 | 0.00134 | 0.00381 | 0.00058 | 0.00390 |
| 4-10 | 0.00161 | 0.00563 | 0.00172 | 0.00534 |
| 10-24 | 0.0244 | 0.00597 | 0.00300 | 0.00482 |
| 24-48 | 0.00305 | 0.00604 | 0.00427 | 0.00616 |
| Time (h) | Std Err | Std Err | Std Err | Std Err |
| 0-4 | 0.00041 | 0.00155 | 0.00010 | 0.00105 |
| 4-10 | 0.00035 | 0.00123 | 0.00022 | 0.00091 |
| 10-24 | 0.00028 | 0.00110 | 0.00045 | 0.00091 |
| 24-48 | 0.0056 | 0.00069 | 0.00092 | 0.00116 |

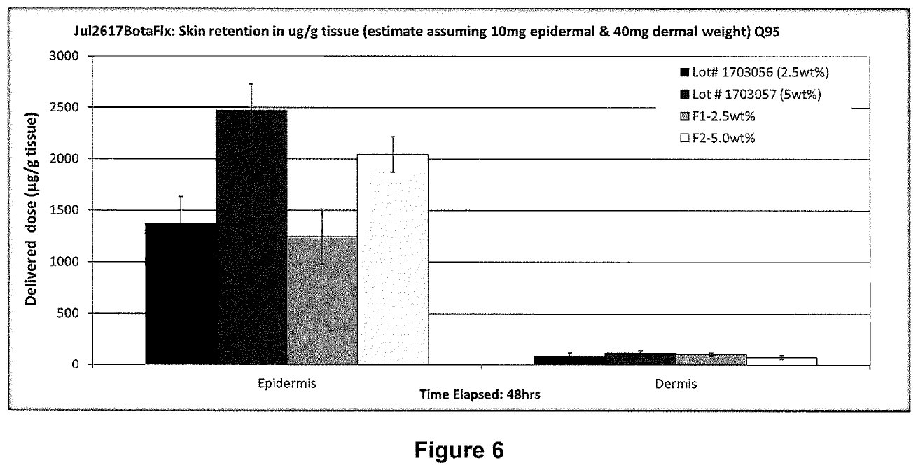

The accumulated dose of CBD in the epidermis and dermis was also calculated as μg of CBD delivered per gram of tissue. This calculation assumes a weight of 10 mg for the epidermal tissue and 40 mg for the dermal tissues (these values are based on average values observed in previous experiments). These values are shown in FIG. 6.

TABLE 13
Total accumulated dose in the skin (in μg/
gram tissue) of CBD delivered at 48 hrs.
Skin Delivery (μg/g)

| | A: 2.5 wt % | B: 5.0 wt % | C: 2.5 wt % | D: 5.0 wt % |
|---|---|---|---|---|
| Time (h) | cannabidiol | cannabidiol | cannabidiol | cannabidiol |
| Epidermis | 1370.66 | 2468.64 | 1245.24 | 2043.60 |
| Dermis | 86.39 | 115.60 | 106.41 | 75.26 |
| Time (h) | Std Err | Std Err | Std Err | Std Err |
| Epidermis | 262.71 | 255.62 | 268.71 | 171.11 |
| Dermis | 30.08 | 27.81 | 14.51 | 19.18 |

A two tailed Ttest with unequal variance was used to evaluate the CBD data sets over time. The Ttest compared the transdermal data sets at 24 hours and 48 hours and the epidermal and dermal values.

TABLE 14
A two-tailed Ttest with unequal variance was done comparing
the CBD data sets at 24 and 48 hrs, plus the epidermal
and dermal concentration (results shown are p-values).

| | | | | |
|---|---|---|---|---|
| | A: 2.5 wt % | B: 5.0 wt % | C: 2.5 wt % | D: 5.0 wt % |
| Formulation | cannabidiol | cannabidiol | cannabidiol | cannabidiol |

T test for 24 h (probability values)

| | | | | |
|---|---|---|---|---|
| A: 2.5 wt % | 1 | | | |
| cannabidiol | | | | |
| B: 5.0 wt % | 0.040 | 1 | | |
| cannabidiol | | | | |
| C: 2.5 wt % | 0.613 | 0.049 | 1 | |
| cannabidiol | | | | |
| D: 5.0 wt % | 0.016 | 0.617 | 0.022 | 1 |
| cannabidiol | | | | |

T Test for 48 h (probability values)

| | | | | |
|---|---|---|---|---|
| A: 2.5 wt % | 1 | | | |
| cannabidiol | | | | |
| B: 5.0 wt % | 0.021 | 1 | | |
| cannabidiol | | | | |
| C: 2.5 wt % | 0.333 | 0.056 | 1 | |
| cannabidiol | | | | |
| D: 5.0 wt % | 0.027 | 0.820 | 0.080 | 1 |
| cannabidiol | | | | |

T Test for Epidermis (probability values)

| | | | | |
|---|---|---|---|---|
| A: 2.5 wt % | 1 | | | |
| cannabidiol | | | | |
| B: 5.0 wt % | 0.013 | 1 | | |
| cannabidiol | | | | |
| C: 2.5 wt % | 0.745 | 0.008 | 1 | |
| cannabidiol | | | | |
| D: 5.0 wt % | 0.062 | 0.201 | 0.035 | 1 |

T Test for Dermis (probability values)

| | | | | |
|---|---|---|---|---|
| A: 2.5 wt % | 1 | | | |
| cannabidiol | | | | |
| B: 5.0 wt % | 0.492 | 1 | | |
| cannabidiol | | | | |
| C: 2.5 wt % | 0.567 | 0.777 | 1 | |
| cannabidiol | | | | |
| D: 5.0 wt % | 0.763 | 0.263 | 0.227 | 1 |
| cannabidiol | | | | |

Based on the results of the Ttest analysis, it was observed that A: 2.5 wt % cannabidiol and B: 5.0 wt % cannabidiol were statistically different at 24 and 48 hrs and in the epidermis with greater than 95% confidence (p-values are 0.040, 0.021, and 0.013 respectively). The dermal values for A: 2.5 wt % cannabidiol and B: 5.0 wt % cannabidiol were not statistically different with a p-value of 0.492.

Based on the results of the Ttest analysis, it was also observed that C: 2.5 wt % cannabidiol and D: 5.0 wt % cannabidiol were statistically different at 24 and 48 hrs and in the epidermis with greater than 90% confidence (p-values are 0.022, 0.080, and 0.035 respectively). The dermal values for C: 2.5 wt % cannabidiol and D: 5.0 wt % cannabidiol were not statistically different with a p-value of 0.227.

Finally, based on the results of the Ttest analysis, there were no statistically significant differences between A: 2.5 wt % cannabidiol and C: 2.5 wt % cannabidiol or between the B: 5.0 wt % cannabidiol and D: 5.0 wt % cannabidiol. These data suggest that there is not a meaningful difference in the flux parameters between the two different CBD formulations.

From the foregoing Examples, it is expected that the use of cannabinoids, such as cannabidiol in accordance with the present invention can deliver increased amounts of cannabidiol into the epidermis and dermis and be used to treat and/or improve the healing of inflammatory skin conditions. Generally, treatment in accordance with the present invention will result in a shortened healing time

1. A pharmaceutical composition comprising a cannabinoid and a siloxane wherein the cannabinoid is dissolved in the composition.

2. The pharmaceutical composition according to claim 1 wherein the cannabinoid is cannabidiol.

3. The pharmaceutical composition according to claim 1 wherein the composition is for topical application.

4. The pharmaceutical composition according to claim 1 wherein the siloxane is selected from the group consisting of: hexamethyldisiloxane, octamethyltrisiloxane and combinations thereof.

5. The pharmaceutical composition according to claim 1 further comprising a residual solvent.

6. The pharmaceutical composition according to claim 5 wherein the residual solvent is selected from the group consisting of: alkyl polypropylene glycol/polyethylene glycol ether (alkyl PEG/PPG ether) and a fatty alcohol.

7. The pharmaceutical composition according to claim 6 wherein the alkyl PEG/PPG ether:
a) has a PEG/PPG chain length of between 10-50 PG units and an ether component of between 2-20 carbons, wherein the sum of the PG units and the carbons of the ether component is between 20 and 60;
b) has a low volatility such that less than 5% would evaporate at skin temperature over 24 hours;
c) is a liquid at about 30° C., or less; and/or
d) is selected from the group consisting of: polypropylene glycol ethers of stearyl alcohol and butyl alcohol.

8. The pharmaceutical composition according to claim 6 wherein the relative amount of alkyl PEG/PPG ether is selected from the following group: at least 1% w/w, at least 2% w/w, at least 3% w/w, at least 4% w/w, and at least 5% w/w.

9. The pharmaceutical composition according to claim 6 wherein the fatty alcohol:
a) has a low volatility such that less than 5% would evaporate at skin temperature over 24 hours;
b) is a C₁₂₋₂₂ fatty alcohol and/or
c) is a liquid at about 30° C., or less.

10. The pharmaceutical composition according to claim 9 wherein the fatty alcohol is selected from the group consisting of: oleyl alcohol, isostearyl alcohol, octyldodecyl alcohol, and 2-hexyl decyl alcohol.

11. The pharmaceutical composition according to claim 1 further comprising a low molecular weight alcohol.

12. The pharmaceutical composition according to claim 11 wherein the low molecular weight alcohol is selected from the group consisting of C₂₋₆ alcohols, and combinations thereof.

13. The pharmaceutical composition according to claim 12 wherein the alcohol is selected from the group consisting of: ethyl alcohol, n-propanol, isopropyl alcohol and combinations thereof.

14. The pharmaceutical composition according to claim 1 characterised in that the concentration of cannabinoid in the topical composition is at least 2% w/w.

15. The pharmaceutical composition according to claim 1 characterised in that the concentration of cannabinoid in the topical composition is at least 20% w/w.

16. A method for treating or preventing an inflammatory skin condition in a patient in need of such treatment, the method comprising topically administering a prophylactically or therapeutically effective amount of a pharmaceutical composition according to claim 1.

17. (canceled)

18. (canceled)
